(19) United States
(12) Patent Application Publication
(10) Pub. No.: US 2024/0293226 A1
(43) Pub. Date: Sep. 5, 2024

(54) PROSTHETIC HEART VALVE DEVICE, SYSTEM, AND METHODS

(71) Applicant: HANGZHOU SEQUOLA MEDICAL DEVICE CO., LTD., Hangzhou (CN)

(72) Inventors: Randy Matthew Lane, Langley (CA); Colin Alexander Nyuli, Vancouver (CA); Zhibin Fu, Hangzhou (CN)

(21) Appl. No.: 18/205,383

(22) Filed: Jun. 2, 2023

Related U.S. Application Data
(63) Continuation of application No. PCT/CN2020/133861, filed on Dec. 4, 2020.

(51) Int. Cl. A61F 2/24

(52) U.S. Cl. CPC A61F 2/2418; A61F 2/2439; A61F 2220/0016; A61F 2250/0036; A61F 2250/0069

(57) ABSTRACT

A system comprised of a prosthetic heart valve device (535, 555, 900, 1108, 1230, 1260, 1400, 1535), and a delivery system (1100, 1105, 1110, 1300, 1500). The prosthetic heart valve device (535, 555, 900, 1108, 1230, 1260, 1400, 1535) comprises a differentially deformable anchoring structure concentrically aligned with, radially adjacent to, and in direct connection with a valve frame (700). The atrial region (805, 1005, 1410, 1805, 1850) of the differentially deformable anchoring structure (800, 1229, 1259) comprises a plurality of alignment structures intended to aid in rotational orientation. This atrial region (805, 1005, 1410, 1805, 1850) is in direct connection with the valve frame through inflow region connection elements. The annular region of the differentially deformable anchoring structure comprises anchoring elements and an architecture having a radial stiffness suitable for deformation and conformation to the native anatomy; this region is void of connection to the valve frame. The ventricular region of the differentially deformable anchoring structure comprises a plurality of ventricular anchoring elements and a plurality of ventricular region connection elements, adjacent to and in contact with the outflow region of the connecting members of the valve frame. The delivery system is comprised of a proximal control assembly connected to a first bendable catheter comprising a primary inner lumen, one or more secondary lumens adjacent to the primary lumen, one or more tethers releasably connected to the atrial portion of the prosthetic heart valve device, and a second elongate catheter with connection elements that are releasably connected to the ventricular portion of the prosthetic heart valve device. A compensation mechanism is in connected communication with the second catheter and controllably enables conformational change of the prosthetic heart valve device during implantation.

### FIELD OF THE INVENTION

The present technology relates generally to prosthetic heart valve devices for repairing and/or replacing native heart valves. In particular, several embodiments are directed to prosthetic atrioventricular valves for replacing defective mitral and/or tricuspid valves, as well as methods and devices for delivering and implanting the same within a human heart.

Certain embodiments disclosed herein relate generally to prostheses for implantation within a lumen or body cavity and delivery systems for a prosthesis. In particular, the prostheses and delivery systems relate in some embodiments to prosthetic heart valve devices, such as replacement atrioventricular valves.

### BACKGROUND OF THE INVENTION

Atrioventricular valve insufficiency, also known as mitral and/or tricuspid valve regurgitation or incompetence, is a heart condition in which the atrioventricular valve (mitral and/or tricuspid) does not close properly. Both the mitral and tricuspid apparati of a healthy human heart are comprised of a fibrous annulus, attached to this are flexible resilient leaflets that close upon ventricular contraction. The free ends of each of the flexible leaflets are attached to chordae tendineae which tether the leaflets to papillary muscles within the ventricle, controlling the motion of the leaflet free ends throughout the cardiac cycle. All these components of the apparati must function in synchrony for proper systemic blood circulation. Various cardiac diseases or degenerative conditions can impact any of the components of an atrioventricular valve, resulting in improper closure of the valve. This results in abnormal leakage of blood flow through the valve into the atrium and peripheral vasculature. Persistent atrioventricular valve regurgitation can result in a myriad of cardiovascular complications, including congestive heart failure.

Traditionally, patients suffering from mitral regurgitation have been treated with invasive open-heart surgery, involving either surgical repair or replacement of the mitral apparatus. Generally, these procedures result in good clinical outcomes, however a large percentage of potential patients do not meet the inclusion criteria for such therapies due to its invasiveness and lengthy recovery periods. Therefore, many patients are left untreated and are managed under medical therapy. Patients suffering from tricuspid regurgitation are treated to an even lesser extent through surgical procedures, therefore an even greater population of medically managed patients suffering from tricuspid regurgitation exist. Patients managed under medical therapy for atrioventricular valve disease can have poor quality of life and unfavorable long-term outcomes; many experiencing a five-year mortality rate of 50% or greater.

Significant advancement in the development of minimally invasive transcatheter valve therapies have been made over the years, with the greatest advancements made in treating aortic and pulmonary valve disease. An exemplary prosthesis includes that described in U.S. Pat. No. 7,892,281; the entire contents of which are incorporated herein by reference in their entirety for all purposes. Some advancement has been made in treating mitral valve insufficiency through transcatheter therapies. An exemplary prosthesis includes that described in U.S. Pat. No. 8,652,203; the entire contents of which are incorporated herein by reference in their entirety for all purposes. An additional exemplary prosthesis includes that described in U.S. Pat. No. 9,034,032; the entire contents of which are incorporated herein by reference in their entirety for all purposes. However, a large population of potential patients remain unsuitable for such therapies and remain untreated or have had unfavorable outcomes due to the limitations of the current technologies. The limitations and outcomes include, but are not limited to, the potential for outflow tract obstruction, thrombus formation and thromboembolic events due to atrial flow stasis and prolonged surgical procedures resulting in adverse events and/or exposed radiation to the patients and surgical staff. Little advancement has been made in treating tricuspid valve insufficiency through transcatheter valve replacement therapies. Given the limitations of the current technologies and the large population of untreated patients, there remains a need for improved devices, systems and methods with greater ease, accuracy, and repeatability for treating atrioventricular valve insufficiency.

### SUMMARY OF THE INVENTION

Embodiments disclosed herein refer to a device, system, and methods; such as but not limited to a replacement prosthetic heart valve device and system for replacement of a deficient atrioventricular valve, more specifically a deficient native tricuspid and/or mitral valve in the heart of a human patient.

Further embodiments are directed to delivery systems, devices and/or methods of use to deliver and/or controllably deploy a prosthetic heart valve device, such as but not limited to a replacement heart valve device, to a desired location within the body.

In some embodiments, a replacement prosthetic heart valve device and methods for delivering a replacement prosthetic heart valve device to a native heart valve, such as an atrioventricular valve, are provided.

The present disclosure includes, but is not limited to, the following numbered embodiments.

### Embodiment 1

A system for replacement of a deficient native atrioventricular valve, comprising a delivery system and a prosthetic heart valve device having two typical operational configurations: a radially compressed operational configuration intended for transcatheter delivery through the intended anatomy, and a radially expanded operational configuration intended for final implantation within the target deficient atrioventricular valve.

### Embodiment 2

The prosthetic heart valve device of embodiment 1, wherein the prosthetic heart valve device can be implanted within a deficient native mitral heart valve, traversing the patient's vasculature from the femoral vein, through the inferior vena cava and the atrial septum to its final implant position within the mitral apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 3

The prosthetic heart valve device of embodiment 1, wherein the prosthetic heart valve device can be implanted within a deficient native tricuspid heart valve, traversing the patient's vasculature from the femoral vein, through the inferior vena cava and right atrium to its final implant position within the tricuspid apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 4

The prosthetic heart valve device of embodiment 1, wherein the prosthetic heart valve device can be implanted within a deficient native mitral heart valve, traversing the patient's vasculature from the subclavian vein, through the superior vena cava to its final implant position within the mitral apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 5

The prosthetic heart valve device of embodiment 1, wherein the prosthetic heart valve device can be implanted within a deficient native tricuspid heart valve, traversing the patient's vasculature from the subclavian vein, through the superior vena cava to its final implant position within the tricuspid apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 6

The prosthetic heart valve device of embodiment 1, wherein the prosthetic heart valve device can be implanted within a deficient native mitral heart valve, traversing the patient's anatomy with a trans-apical approach, through the left ventricle to its final implant position within the mitral apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 7

The prosthetic heart valve device of embodiment 1, wherein the prosthetic heart valve device can be implanted within a deficient native tricuspid heart valve, traversing the patient's anatomy with a trans-apical approach, through the right ventricle to its final implant position within the tricuspid apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 8

The prosthetic heart valve device of embodiment 1, wherein the prosthetic heart valve device can be implanted within a deficient native mitral heart valve, traversing the patient's anatomy with a trans-atrial approach, through the left atrium to its final implant position within the mitral apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 9

The prosthetic heart valve device of embodiment 1, wherein the prosthetic heart valve device can be implanted within a deficient native mitral heart valve, traversing the patient's anatomy with a trans-aortic approach, through the femoral artery and aorta to its final implant position within the mitral apparatus, whereby in this exemplary embodiment, the prosthetic heart valve device can be delivered to the intended implant location utilizing a delivery catheter with controlled deployment steps to ensure accurate alignment, placement, and securement of the prosthetic heart valve device.

### Embodiment 10

The prosthetic heart valve device of any one of embodiments 2 through 9, wherein the prosthetic heart valve device may be comprised of a differentially deformable anchoring structure concentrically aligned with, radially adjacent to, in direct connection to and surrounding a valve frame.

### Embodiment 11

The prosthetic heart valve device of embodiment 10, wherein the differentially deformable anchoring structure is comprised of an atrial region having a first stiffness and a plurality of alignment structures intended to aid in rotational orientation during implantation.

### Embodiment 12

The prosthetic heart valve device of embodiment 11, wherein the atrial region is configured to conform to the floor of a native atrium adjacent an atrioventricular valve and can be in direct connection with the internal valve frame through inflow region connection members.

### Embodiment 13

The prosthetic heart valve device of embodiment 12, wherein the differentially deformable anchoring structure comprises an annular region, generally having a second stiffness suitable for deformation and conformation to the native anatomy in addition to comprising annular anchoring elements for preventing retrograde migration.

### Embodiment 14

The prosthetic heart valve device of embodiment 13, wherein the differentially deformable anchoring structure comprises a ventricular region generally having a third stiffness and comprising a plurality of ventricular anchoring elements having a plurality of ventricular region connection elements, adjacent to and in contact with the outflow region of the connecting members of the valve frame.

### Embodiment 15

The prosthetic heart valve device of embodiment 14, wherein the differentially deformable anchoring structure is further configured to be covered by a leakage prevention membrane in both the atrial region and the annular region, to prevent paravalvular leakage.

### Embodiment 16

The prosthetic heart valve device of embodiment 15, wherein the prosthetic heart valve device further comprises a valve frame.

### Embodiment 17

The prosthetic heart valve device of embodiment 16, wherein the valve frame comprises an inflow region, a mid region and an outflow region downstream of the inflow region.

### Embodiment 18

The prosthetic heart valve device of embodiment 17, wherein the inflow region of the valve frame is further configured to be in direct connection with the atrial region of the differentially deformable anchoring structure through inflow region connection members.

### Embodiment 19

The prosthetic heart valve device of embodiment 18, wherein the connection members further comprise flexure geometry configured to mechanically dampen the transmission of forces and distortions from the anchoring structure to the valve frame, while maintaining a secure connection therebetween, and allowing the valve frame to remain in its generally cylindrical geometry for optimized valve performance.

### Embodiment 20

The prosthetic heart valve device of embodiment 19, wherein the inflow region of the valve frame is further configured to contain a leakage prevention membrane which spans from the valve frame to the anchor structure along the connection members.

### Embodiment 21

The prosthetic heart valve device of embodiment 20, wherein the mid region of the valve frame further comprises a plurality of leaflets supported by a leaflet support structure extending throughout the mid region of the valve frame body, in addition to a leakage prevention membrane, which collectively form a one-way valve for the flow of blood through the prosthetic valve assembly.

### Embodiment 22

The prosthetic heart valve device of embodiment 21, wherein the outflow region of the valve frame further comprises a plurality of outflow region connection members in direct connection with the ventricular region of the anchor structure, and wherein the outflow region connection members extend from a commissural region of the valve frame.

### Embodiment 23

The prosthetic heart valve device of embodiment 22, wherein the outflow region connection members further comprise a flexure geometry configured to mechanically dampen the transmission of force between the anchoring structure and the valve frame.

### Embodiment 24

The prosthetic heart valve device of embodiment 23, wherein the flexure geometry further comprises suture-like filaments having a resilience or stretchiness that can range from relatively stiff to relatively flexible.

### Embodiment 25

The prosthetic heart valve device of embodiment 24, wherein the prosthetic heart valve device is further configured for aligning any leaflet of the prosthetic valve with the anterior leaflet of the native atrioventricular valve during implantation, in order to avoid ventricular outflow tract obstruction, by way of guided rotational orientation of the atrial alignment structures within the differentially deformable anchoring structure

### Embodiment 26

The prosthetic heart valve device of embodiment 25, wherein the flexure geometry contained within the inflow region and outflow regions of the valve frame is further configured to allow for cyclic shuttling of the valve prosthesis.

### Embodiment 27

The prosthetic heart valve device of embodiment 26 wherein the flexure geometry within the valve frame is configured to allow for the displacement of the internal prosthetic valve towards the atrium, thereby displacing it from potentially obstructing the ventricular outflow tract and optimizing ventricular output when upon systolic contraction of the ventricle an increase in ventricular pressure displaces the prosthetic valve leaflets from the open to the closed position, increasing the backpressure on the valve.

### Embodiment 28

The prosthetic heart valve device of embodiment 27, wherein upon ventricular expansion, as the differential pressure between the atrium and ventricle is reduced, blood is allowed to flow from the atrium through the prosthetic valve and into the ventricle for ventricular filling and the flexure geometry within the internal valve frame is further configured to allow the valve frame to return to its original position within the ventricular cavity, reducing its atrial projection, reducing the potential for diastolic flow obstruction, blood stasis, and optimizing ventricular filling.

### Embodiment 29

The prosthetic heart valve device of embodiment 28, wherein the radially compressed prosthetic heart valve device further allows for advancement along anatomical routes demanding the traversal of tight tortuous curvature, without anatomical compromise.

### Embodiment 30

The prosthetic heart valve device of embodiment 29, wherein the radially compressed prosthetic heart valve device is delivered in articulated segments.

### Embodiment 31

The prosthetic heart valve device of embodiment 30, wherein the radially compressed prosthetic heart valve device further comprises flexible geometric regions.

### Embodiment 32

The prosthetic heart valve device of embodiment 31, wherein the differentially deformable anchoring structure allows for optimized control of advancement and delivery of the prosthetic heart valve device to the intended target implant site, by providing allowance for longer compressed prosthetic heart valve devices being advanced along tortuous routes.

### Embodiment 33

The delivery system of embodiment 32, wherein the delivery system comprises an elongate first catheter having a first diameter and comprising a primary lumen, a first bendable portion, and one or more secondary lumens radially adjacent to the primary lumen.

### Embodiment 34

The delivery system of embodiment 33, further comprising one or more tethers that are connectable to a portion of the prosthetic heart valve device and configured to translate through the one or more secondary lumens of the first catheter.

### Embodiment 35

The delivery system of embodiment 34, further comprising an elongate second catheter having a second diameter smaller than the first diameter and comprising a lumen, a second bendable portion, and one or more connection elements that are connectable to a portion of the prosthetic heart valve device; wherein the second catheter is further configured to translate within the primary lumen of the first catheter.

### Embodiment 36

The delivery system of embodiment 35, further comprising a compensation mechanism that is in connected communication with the second catheter and that controllably enables conformational change of the prosthetic heart valve device.

### Embodiment 37

The delivery system of embodiment 36, wherein the one or more tethers and the one or more connection elements collectively provide tensile force which controllably maintains the prosthetic heart valve device in a radially restrained configuration for delivery.

### Embodiment 38

The delivery system of embodiment 37, wherein the compensation mechanism allows the second catheter to release tensile force by controllably translating within the first catheter during radial expansion of the prosthetic heart valve device.

### Embodiment 39

The delivery system of embodiment 38, further comprising an elongate third catheter having a third diameter smaller than the second and comprising a lumen, a third bendable portion, and a distal covering having a fourth diameter larger than the third diameter and configured to radially restrain a portion of the prosthetic heart valve device by containing a portion of it therein.

### Embodiment 40

The delivery system of embodiment 39, wherein the third catheter is further configured to translate within the lumen of the second catheter.

### Embodiment 41

The delivery system of embodiment 40, wherein the distal covering is further configured to entrap a portion of the prosthetic heart valve device through contact with the connection elements of the second catheter.

### Embodiment 42

The delivery system of embodiment 41, wherein the compensation mechanism is further configured to be in connected communication with the third catheter, and wherein the distal covering of the third catheter is controllably translated by actuation of the compensation mechanism.

### Embodiment 43

The delivery system of embodiment 42, further comprising a fourth elongate catheter having a fifth diameter larger than the first diameter and comprising a lumen and a proximal covering configured to support radially restraining a portion of the prosthetic heart valve device by containing a portion of it therein

### Embodiment 44

The delivery system of embodiment 43, wherein the fourth catheter is further configured to translate overtop the first catheter.

### Embodiment 45

The delivery system of embodiment 44, wherein the first and second bendable portions further comprise a portion of laser-cut nitinol tubing.

### Embodiment 46

The delivery system of embodiment 44, wherein the first and second bendable portions further comprise a portion of laser-cut steel tubing.

### Embodiment 47

The delivery system of embodiment 44, wherein the first and second bendable portions further comprise a portion of laser-cut polymer tubing.

### Embodiment 48

The delivery system of embodiment 44, wherein the first and second bendable portions further comprise a portion of reinforced fibre tubing.

### Embodiment 49

The delivery system of any of embodiments 45-48, wherein the second catheter is further configured to be steerable by way of the application of tensile force to internally biased pull-wires.

The present invention will be more fully understood from the following detailed description of applications thereof, taken together with the drawings, in which:

### BRIEF DESCRIPTION OF THE DRAWINGS

FIG. 1 is a schematic illustration of a front view of an anterior aspect of an exemplary heart, in accordance with some applications of the invention.

FIG. 2A is a schematic illustration of a front view of a posterior aspect of an exemplary heart having section lines, in accordance with some applications of the invention.

FIG. 2B is a schematic illustration of a sectioned view of a basal aspect of an exemplary heart, showing an exemplary aortic valve, an exemplary mitral valve, an exemplary pulmonary valve, and an exemplary tricuspid valve, in accordance with some applications of the invention.

FIG. 3A is a schematic illustration of a front view of an unfurled and flattened perimeter of an exemplary native mitral apparatus including leaflets, chordae tendineae and papillary muscles, in accordance with some applications of the invention.

FIG. 3B is a schematic illustration of a front view of an unfurled and flattened perimeter of an exemplary native tricuspid apparatus including leaflets, chordae tendineae and papillary muscles, in accordance with some applications of the invention.

FIG. 4A is a schematic illustration of a sectioned view of an anterior aspect of an exemplary heart, showing the direction of normal blood flow in the left ventricle, during diastole in accordance with some applications of the invention.

FIG. 4B is a schematic illustration of a sectioned view of an anterior aspect of an exemplary heart, showing the direction of normal blood flow in the left ventricle, during systole in accordance with some applications of the invention.

FIG. 4C is a schematic illustration of a sectioned view of an anterior aspect of an exemplary heart, showing the direction of regurgitant blood flow in the left ventricle due to a flail posterior leaflet, during systole in accordance with some applications of the invention.

FIG. 4D is a schematic illustration of a sectioned view of an anterior aspect of an exemplary heart, showing the direction of regurgitant blood flow in the left ventricle due to leaflet tenting, during systole in accordance with some applications of the invention.

FIG. 5A is a schematic illustration of a sectioned view of an anterior aspect of an exemplary heart, showing an embodiment of a prosthetic heart valve device implanted within the mitral position in accordance with some applications of the invention.

FIG. 5B is a schematic illustration of a sectioned view of an anterior aspect of an exemplary heart, showing an embodiment of a prosthetic heart valve device implanted within the tricuspid position, in accordance with some applications of the invention.

FIG. 6A is a schematic illustration of a sectioned view of an anterior aspect of an exemplary heart, showing the percutaneous pathway corresponding to transapical implantation within the mitral position, in accordance with some applications of the invention.

FIG. 6B is a schematic illustration of a sectioned view of an anterior aspect of an exemplary heart, showing the percutaneous pathway corresponding to transapical implantation within the tricuspid position, in accordance with some applications of the invention.

FIG. 6C is a schematic illustration of a sectioned view of an anterior aspect of an exemplary heart, showing the percutaneous pathway corresponding to transfemoral venous implantation within the tricuspid position, in accordance with some applications of the invention.

FIG. 6D is a schematic illustration of a sectioned view of an anterior aspect of an exemplary heart, showing the percutaneous pathway corresponding to transseptal implantation within the mitral position, in accordance with some applications of the invention.

FIG. 6E is a schematic illustration of a sectioned view of an anterior aspect of an exemplary heart, showing the percutaneous pathway corresponding to transsubclavian implantation within the mitral position, in accordance with some applications of the invention.

FIG. 6F is a schematic illustration of a sectioned view of an anterior aspect of an exemplary heart, showing the percutaneous pathway corresponding to transsubclavian implantation within the tricuspid position, in accordance with some applications of the invention.

FIG. 6G is a schematic illustration of a sectioned view of an anterior aspect of an exemplary heart, showing the percutaneous pathway corresponding to transaortic implantation within the mitral position, in accordance with some applications of the invention.

FIG. 6H is a schematic illustration of a sectioned view of an anterior aspect of an exemplary heart, showing the percutaneous pathway corresponding to transatrial implantation within the mitral position, in accordance with some applications of the invention.

FIG. 7A is a schematic illustration of a perspective view of an embodiment of an exemplary self expanding valve frame, in accordance with some applications of the invention.

FIG. 7B is a schematic illustration of an overhead (inflow) view of an embodiment of an exemplary self expanding valve frame, in accordance with some applications of the invention.

FIG. 7C is a schematic illustration of a front view of an embodiment of an exemplary self expanding valve frame, in accordance with some applications of the invention.

FIG. 7D is a schematic illustration of a front view of an embodiment of an exemplary self expanding valve frame, including tissue leaflets and fabric coverings, in accordance with some applications of the invention.

FIG. 8A is a schematic illustration of a perspective view of an embodiment of an exemplary differentially deformable anchoring structure, in accordance with some applications of the invention.

FIG. 8B is a schematic illustration of a profile view of an embodiment of an exemplary differentially deformable anchoring structure, in accordance with some applications of the invention.

FIG. 8C is a schematic illustration of an overhead (inflow) view of an embodiment of an exemplary differentially deformable anchoring structure, in accordance with some applications of the invention.

FIG. 8D is a schematic illustration of a profile view of an embodiment of an exemplary differentially deformable anchoring structure, including fabric coverings, in accordance with some applications of the invention.

FIG. 9A is a schematic illustration of a front view of an embodiment of an exemplary prosthetic heart valve device, in accordance with some applications of the invention.

FIG. 9B is a schematic illustration of a perspective view of an embodiment of an exemplary prosthetic heart valve device, in accordance with some applications of the invention.

FIG. 9C is a schematic illustration of a perspective overhead (inflow) view of an embodiment of an exemplary prosthetic heart valve device, in accordance with some applications of the invention.

FIG. 9D is a schematic illustration of a front view of an embodiment of an exemplary prosthetic heart valve device, including fabric coverings, in accordance with some applications of the invention.

FIG. 9E is a schematic illustration of a cross-sectional profile view of an embodiment of an exemplary prosthetic heart valve device, in accordance with some applications of the invention.

FIG. 9F is a schematic illustration of an embodiment of an exemplary prosthetic heart valve device, detailing alternative embodiments of flexure geometry connection.

FIG. 10A is a schematic illustration of a front view of an embodiment of an exemplary prosthetic heart valve device in a crimped configuration, in accordance with some applications of the invention.

FIG. 10B is a schematic illustration of a front view of an embodiment of an exemplary prosthetic heart valve device in an expanded configuration, in accordance with some applications of the invention.

FIG. 11A is a schematic illustration of a front view of an embodiment of an exemplary prosthetic heart valve device being deployed from an exemplary delivery system, in accordance with some applications of the invention.

FIG. 11B is a schematic illustration of a front view of an embodiment of an exemplary prosthetic heart valve device being deployed from an exemplary delivery system, in accordance with some applications of the invention.

FIG. 11C is a schematic illustration of a front view of an embodiment of an exemplary prosthetic heart valve device being deployed from an exemplary delivery system, in accordance with some applications of the invention.

FIG. 12A is a schematic illustration of a side sectioned view of an embodiment of an exemplary prosthetic heart valve device implanted within the mitral position, in the diastolic phase of the cardiac cycle, in accordance with some applications of the invention.

FIG. 12B is a schematic illustration of a side sectioned view of an embodiment of an exemplary prosthetic heart valve device implanted within the mitral position, in the systolic phase of the cardiac cycle, in accordance with some applications of the invention.

FIG. 13A is a schematic illustration of a perspective view with a detailed view of an embodiment of an exemplary prosthetic heart valve device loaded into an exemplary delivery system, in accordance with some applications of the invention.

FIG. 13B is a schematic illustration of a front view of a segment of an embodiment of an exemplary prosthetic heart valve device frame flat pattern, in accordance with some applications of the invention.

FIG. 14 is a schematic illustration of an enlarged view of a distal portion of a transfemoral delivery device with a prosthesis in a partially deployed configuration, in accordance with some applications of the invention.

FIG. 15A is a schematic illustration of a transfemoral delivery device with a prosthetic heart valve device in a loaded configuration, in accordance with some applications of the invention.

FIG. 15B is a schematic illustration of a distal portion of a transfemoral delivery device with a prosthetic heart valve device in a loaded configuration, in accordance with some applications of the invention.

FIG. 16A is a schematic illustration of a transfemoral delivery device, in accordance with some applications of the invention.

FIG. 16B is a schematic illustration of a transfemoral delivery device, in accordance with some applications of the invention.

FIG. 17A is a schematic illustration of a prosthetic heart valve device retention region of a transfemoral delivery device, in accordance with some applications of the invention.

FIG. 17B is a schematic illustration of a tether shuttling mechanism of a transfemoral delivery device, with tether shuttles in a closed configuration, in accordance with some applications of the invention.

FIG. 17C is a schematic illustration of a plurality of tether connectors of a transfemoral delivery device, in an engaged configuration and in accordance with some applications of the invention.

FIG. 17D is a schematic illustration of a tether shuttling mechanism of a transfemoral delivery device, with tether shuttles in an opened configuration, in accordance with some applications of the invention.

FIG. 17E is a schematic illustration of a plurality of tether connectors of a transfemoral delivery system, in a disengaged configuration, in accordance with some applications of the invention.

FIG. 17F is a schematic illustration of a tether connector of a transfemoral delivery system, in a hidden-line view, in accordance with some applications of the invention.

FIGS. 18A-I are a sequence of schematic illustrations depicting the deployment of a prosthetic heart valve device, in accordance with some applications of the invention.

FIGS. 19A-D are a sequence of schematic illustrations depicting the conformational mechanics of a second catheter and an outer covering at the retention region, in accordance with some applications of the invention.

FIGS. 20A-C are a series of schematic illustrations of a transfemoral delivery device depicted in cross-section, in accordance with some applications of the invention.

### DETAILED DESCRIPTION OF THE PREFERRED EMBODIMENTS

The present specification and drawings provide aspects and features of the disclosure in the context of several embodiments of replacement prosthetic heart valve devices, systems, and methods that are configured for use in the vasculature of a patient, such as for replacement of native heart valves in a patient. These embodiments may be discussed in connection with replacing specific valves such as the patient's mitral or tricuspid valve. However, it is to be understood that the features and concepts discussed herein can be applied to products other than prosthetic heart valve devices. For example, the controlled positioning, deployment, and securing features described herein may be applied to medical implants, for example other types of expandable prosthesis, for use elsewhere in the body, such as within an artery, a vein, or other body cavities or locations. In addition, particular features of a prosthetic heart valve device, system, or methods should not be taken as limiting, and features of any one embodiment discussed herein may be combined with features of other embodiments as desired and when appropriate. While certain of the embodiments described herein are described in connection with a specific delivery approach, it should be understood that these embodiments may be used for other delivery approaches. Moreover, it should be understood that certain of the features described in connection with some embodiments can be incorporated with other embodiments, including those which are described in connection with different delivery approaches.

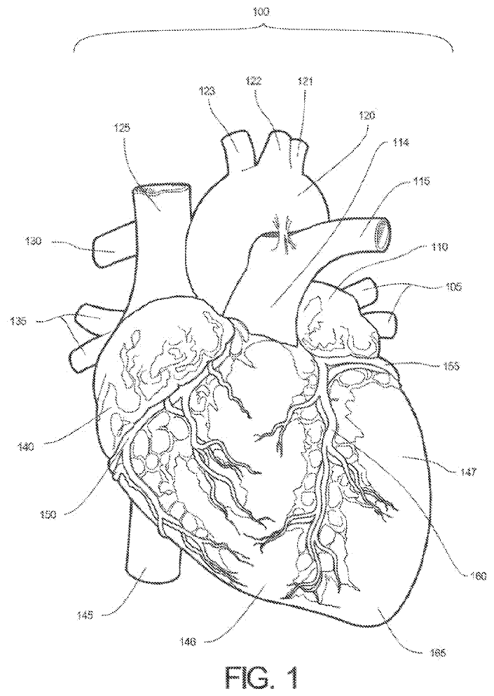

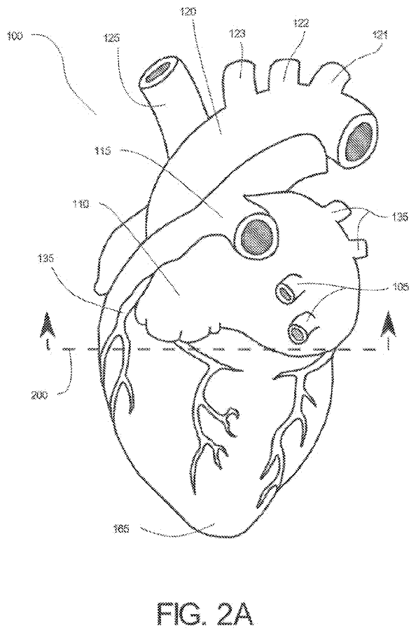

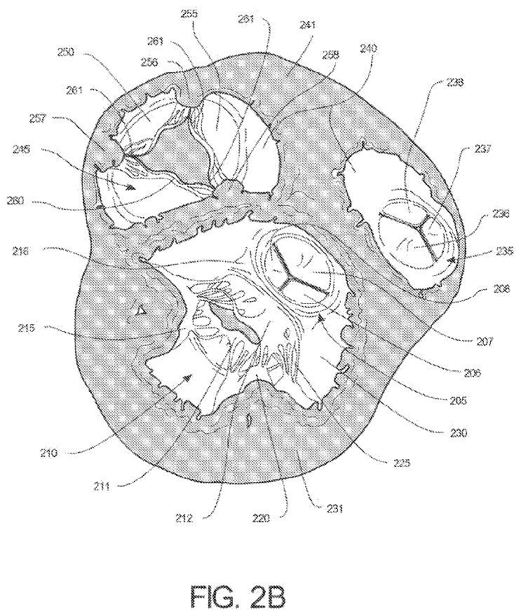

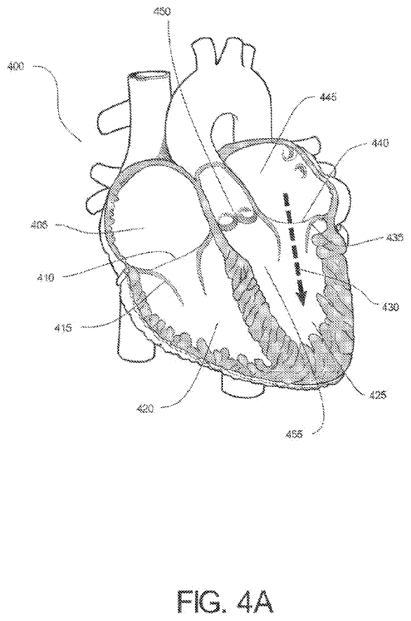

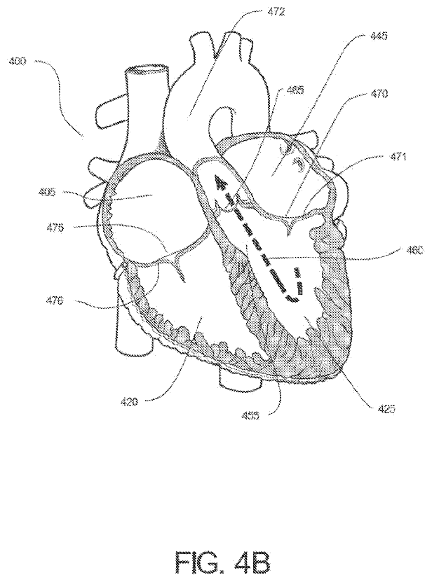

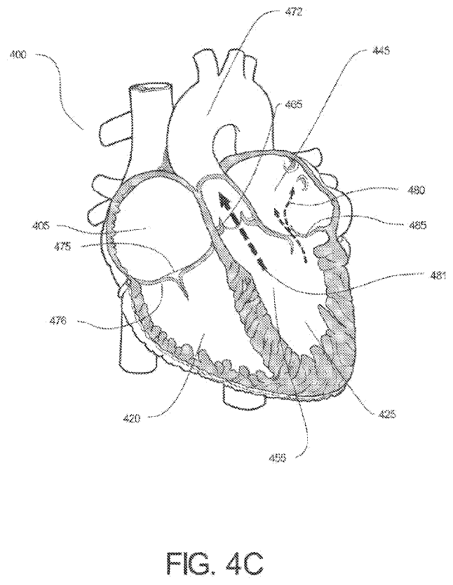

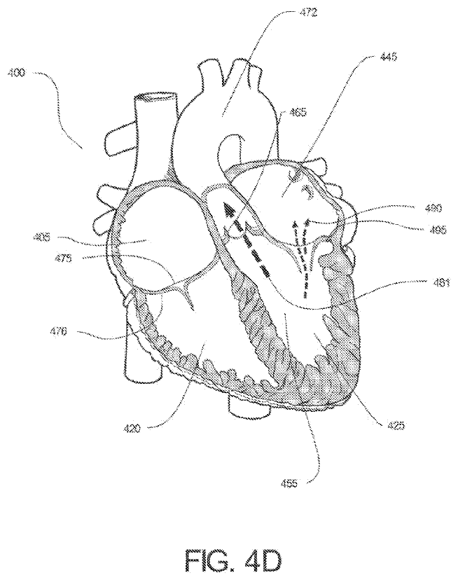

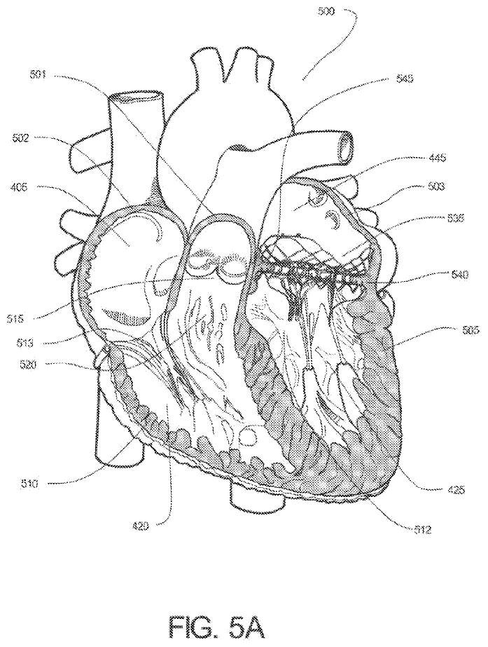

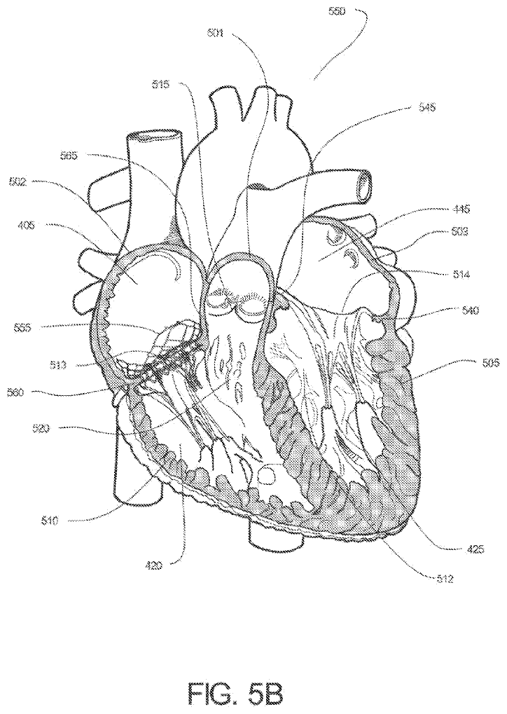

Reference is made to FIG. 1, which is a schematic illustration showing a front view of an anterior aspect of an exemplary heart 100, in accordance with some applications of the invention. The exemplary heart 100 is generally comprised of four main chambers (right atrium 140, right ventricle 146, left atrium 110 and left ventricle 147), which act harmoniously as a pumping system to circulate blood throughout the vascular system. Normally, the systemic circulation (not shown) returns deoxygenated blood through the superior and inferior vena cava (125, 145 respectively) to the right atrium 140. During diastole (ventricular expansion portion of the cardiac cycle), the deoxygenated blood is forced through the tricuspid valve (245, FIG. 2B) and into the right ventricle 146. Once in the right ventricle 146, a systolic (ventricular contraction portion of the cardiac cycle) contraction driven pressure gradient between the right ventricle 146 and right atrium 140 closes the tricuspid valve (245, FIG. 2B) and forces blood through the right ventricular outflow tract (520, FIG. 5A), through the pulmonary valve (515, FIG. 5A) and along the pulmonary trunk 114 until it exits towards the lungs (not shown) by traveling along the left and right pulmonary arteries (115, 130 respectively). The blood becomes oxygenated through respiration by the lungs (not shown) and is then returned through the left and right pulmonary veins (105, 135 respectively) into the left atrium 110. A diastolic expansion then draws the now oxygenated blood through the open mitral valve (210, FIG. 2B), resulting in left ventricular 147 filling. Finally, systolic ventricular contraction drives a pressure gradient between the left ventricle 147 and the left atrium 110, closing the mitral valve (210, FIG. 2B) and forcing the oxygenated blood within the left ventricle of the heart 147 through the left ventricular outflow tract (455, FIG. 4A), through the aortic valve (205, FIG. 2B), and along the aorta 120 to the systemic circulation (not shown). The heart 100 also provides itself with oxygenated blood throughout the cardiac cycle, by way of the circumflex artery 155, and the left and right coronary arteries (160, 150 respectively). Branching arteries of the aorta 120 such as the left subclavian, left common carotid, and brachiocephalic (121, 122, 123 respectively) provide oxygenated blood to the brain and upper extremities of the body.

Turning now, reference is made to FIG. 2A, which is a schematic illustration of a posterior aspect of an exemplary heart 100, in accordance with some applications of the invention. Section line A-A 200 is shown, which illustrates where a section may be cut through the exemplary heart 100 to arrive at the view depicted in FIG. 2B.

FIG. 2B is a schematic illustration showing a sectioned view of the exemplary heart 100, highlighting the anatomical features presented when viewed from an apical perspective, in accordance with some applications of the invention. As described previously, the exemplary heart is generally comprised of four main chambers (right atrium 140, right ventricle 146, left atrium 110 and left ventricle 147, FIG. 1); between the right atrium (140, FIG. 1) and right ventricle (146, FIG. 1) is found the tricuspid valve 245. The inner wall of the right ventricle 240 defines a space in which blood is pumped from during systolic contraction. The tricuspid valve 245 is a tri-leaflet valve, and is comprised of an anterior cusp 255, a posterior cusp 250, and a septal cusp 260 which close together and normally prevent retrograde blood-flow when the right ventricle (146, FIG. 1) becomes pressurized during systole. Between and inferior to the anterior 255 and posterior 250 cusps are found the antero-posterior papillary muscle 256, which supports both leaflets with tricuspid chordae tendineae 261. Between and inferior to the posterior 250 and septal 260 cusps are found the postero-septal papillary muscle 257, which supports both leaflets with tricuspid chordae tendineae 261. Between and inferior to the septal 260 and anterior 255 cusps are found the antero-septal papillary muscle 258, which supports both leaflets with tricuspid chordae tendineae 261.

Following the outer wall of the right ventricle 241 leads to the pulmonary valve 235, which shares the right ventricle (146, FIG. 1) and right ventricular outflow tract (520, FIG. 5A) with the tricuspid valve 245. The pulmonary valve 235 is also a tri-leaflet valve and is comprised of a left cusp 236, a right cusp 238, and an anterior cusp 237, which close together and normally prevent retrograde blood-flow when the right ventricle (146, FIG. 1) becomes de-pressurized during diastole.

Following the outer wall of the left ventricle 231 leads to the aortic valve 205, which shares the left ventricle (147, FIG. 1) and the left ventricular outflow tract (455, FIG. 4A) with the mitral valve 210. The aortic valve 205 is also a tri-leaflet valve and is comprised of a left cusp 206, a right cusp 207, and a posterior cusp 208, which close together and normally prevent retrograde blood-flow when the left ventricle (147, FIG. 1) becomes de-pressurized during systole.

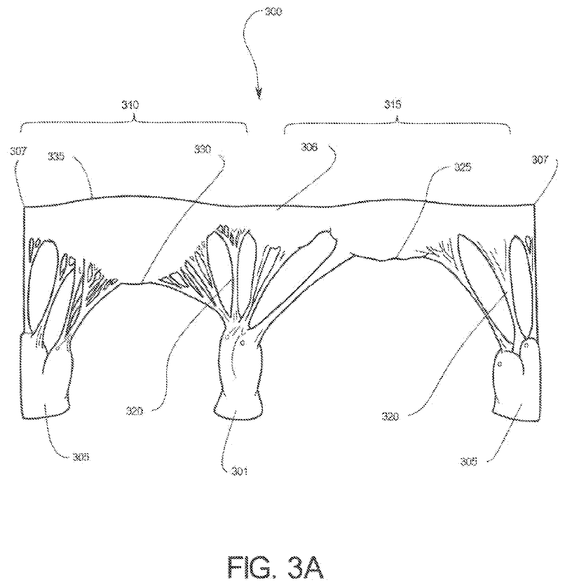

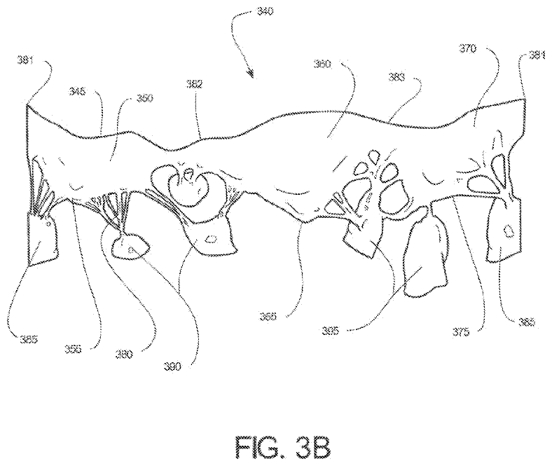

Between the left atrium (110, FIG. 1) and left ventricle (147, FIG. 1) is found the mitral valve 210. The inner wall of the left ventricle 230 defines a space in which blood is pumped from during systolic contraction. The mitral valve 210 is a bi-leaflet valve, and is comprised of an anterior cusp 212, and a posterior cusp 211 which close together and normally prevent retrograde blood-flow when the left ventricle (147, FIG. 1) becomes pressurized during systole. Medial and inferior to the posterior 211 and anterior 212 cusps are found the postero-medial papillary muscle 215, which supports both leaflets with mitral chordae tendineae 225. Lateral and inferior to the posterior 211 and anterior 212 cusps are found the antero-lateral papillary muscle 220, which supports both leaflets with mitral chordae tendineae 225. The anterior cusp 212 extends sub annularly into the ventricle from the mitral annulus (335, FIG. 3A). At the commissural edges (corners where cusps meet) the anterior cusp 212 originates at the annulus near distinctly rigid regions of fibrous tissue knowns as fibrous trigones 216. The fibrous trigones 216 act as structural regions of the heart 100, providing a base of support for the mitral valve 210 and aortic valve 205 during the dynamic motions generated throughout the cardiac cycle.

Reference is now made to FIG. 3A, which is a schematic illustration of a front view of an unfurled and flattened alternative representation 300 of the perimeter of an exemplary native mitral apparatus including leaflets (anterior 310, posterior 315), mitral chordae tendineae (320), and papillary muscles (antero-lateral 305, postero-medial 301) in accordance with some applications of the invention. It can be seen that both the anterior leaflet 310 and posterior leaflet 315 originate at the mitral annulus 335 and extend downwardly (towards the left ventricle, not shown) and away from the left atrium (not shown). Dividing the representation 300 into segments along the edge of the mitral annulus 335 are the postero-medial commissure region 306, and the antero-lateral commissure region 307 (split into two halves within this view). Extending below each commissure region (postero-medial 306, antero-lateral 307) is an arcade of mitral chordae tendineae 320, which further extend into communication with a respective papillary muscle (postero-medial 301, antero-lateral 305). The mitral chordae tendineae also extend directly from the anterior 310 and posterior 315 leaflets themselves, defining the edge of each respective leaflet up until chordae-free regions known as the posterior and anterior free margins (325, 330 respectively) are reached. In a healthy heart with uncompromised anatomy, the function of the chordae tendineae are to provide tension between leaflets and papillary muscles, preventing the leaflets from over-coapting and moving towards the atrium during systole, which could eventually lead to valve dysfunction, regurgitant blood flow, heart failure, and poor health.

Similarly to FIG. 3A, FIG. 3B is a schematic illustration of a view of an unfurled and flattened alternative representation 340 of the perimeter of an exemplary native tricuspid apparatus including leaflets (septal 350, anterior 360, posterior 370), tricuspid chordae tendineae (380), and papillary muscles (postero-septal 385, antero-septal 390, antero-posterior 395), in accordance with some applications of the invention. It can be seen that the anterior 360, posterior 370, and septal 350 leaflets originate at the tricuspid annulus 345 and extend downwardly (towards the right ventricle, not shown) and away from the right atrium (not shown). Dividing the representation 340 into segments along the edge of the tricuspid annulus 345 are the antero-septal commissure region 382, and the antero-posterior commissure region 383, and the postero-septal commissure region 381 (split into two halves within this view). Extending below each commissure region (antero-septal 382, antero-posterior 383, and postero-septal 381) is an arcade of tricuspid chordae tendineae 380, which further extend into communication with a respective papillary muscle (antero-septal 390, antero-posterior 395, and postero-septal 385). The tricuspid chordae tendineae 380 also extend directly from the septal 350, anterior 360, and posterior 370 leaflets themselves, defining the edge of each respective leaflet up until chordae-free regions known as the septal, anterior, and posterior free margins (355, 365, 375 respectively) are reached. As with the mitral valve, the leaflets, chordae tendineae, and respective papillary muscles of the tricuspid valve also function harmoniously, preventing retrograde and regurgitant blood-flow as well as all of the associated diseases and co-morbidities related to said regurgitation.

Reference is now made to FIGS. 4A and 4B, which are schematic illustrations showing the typical depiction of normal forward blood-flow, through the cardiac cycle and including the stages of diastole and systole, for both the left and right sides of the heart (focusing on the left side) in accordance with some aspects of the invention. Specifically, FIG. 4A schematically illustrates a sectioned view of an anterior aspect of an exemplary heart 400, showing the direction of normal blood flow (represented by arrow 430) from the left atrium 445 to the left ventricle 425, during diastole. It will be recognized that during diastole the mitral valve 440 is open, the mitral valve leaflets 435 being fully extended towards the left ventricle 425 in order to allow freshly oxygenated blood to fill said left ventricle 425. During diastole, the aortic valve 450 remains closed. Also depicted in FIG. 4A is the right side of the heart during diastole. In a similar fashion to what occurs in the left side of the heart during diastole, within the right side, blood is directed from the right atrium 405 through the open tricuspid valve 410, past the fully extended tricuspid leaflets 415 and into the right ventricle 420, prior to being driven out of the right ventricular outflow tract (not illustrated) and out the pulmonary valve and further, the pulmonary trunk (neither illustrated). During the cardiac cycle, both ventricles of the heart will expand in unison in diastole, prior to both contracting in unison in systole. FIG. 4B schematically illustrates a sectioned view of an anterior aspect of an exemplary heart 400, showing the direction of normal blood flow (represented by arrow 460) from the left ventricle 425 through the left ventricular outflow tract 455, and towards the aortic valve 465 during systole. It will be recognized that during systole the mitral valve 470 is closed, the mitral valve leaflets 471 being fully collapsed to prevent retrograde blood-flow towards the left atrium 445, and to allow freshly oxygenated blood to be ejected through the aorta 472. During systole, the aortic valve 465 is forced open. Also schematically illustrated in FIG. 4B is the right side of the heart during systole. In a similar fashion to what occurs in the left side of the heart during systole, within the right side, blood is directed from the right ventricle 420 through the right ventricular outflow tract (not shown), and towards the pulmonary valve (not shown). It can be seen that the tricuspid valve 475 is closed, and the tricuspid valve leaflets 476 are fully collapsed to prevent retrograde blood-flow towards the right atrium 405.

In contrast to FIGS. 4A and 4B, FIGS. 4C and 4D schematically illustrate the typical depiction of abnormal forward blood-flow with a portion of retrograde regurgitant flow during the stage of systole, for both the left and right sides of the heart (focusing on the left side), in accordance with some applications of the invention. Specifically, FIG. 4C schematically illustrates a sectioned view of an anterior aspect of an exemplary heart 400, showing the direction of abnormal blood flow (represented by arrows 480 and 481) both through the aorta 465, and back through a compromised mitral valve 485 and into the left atrium 445 during systole. In this illustration, the compromised mitral valve 485 suffers from flailing leaflets that no longer coapt properly. Flailing leaflets may be caused by snapped chordae (not shown), or degenerated mitral annular tissues, which can lead to further tissue structural compromise, reduced strength, and degradation. With this type of compromised mitral valve 485, a significant portion of the ejection fraction that would normally exit through the aorta 465 will be redirected back towards the left atrium 445, as depicted by arrows 480. FIG. 4D schematically illustrates a sectioned view of an anterior aspect of an exemplary heart 400, showing the direction of abnormal blood flow (represented by arrows 490 and 481) both through the aorta 465, and back through a compromised mitral valve 495 and into the left atrium 445 during systole. In this illustration, the compromised mitral valve 495 suffers from tented leaflets that no longer coapt properly. Tented leaflets may be caused by ventricular remodeling, which may happen after an ischemic event such as a heart attack. When a portion of the ventricle loses function (due to ischemia), the remaining healthy portions of the ventricle are forced to over-contract, leading to localized hypertrophy and distortion of surrounding anatomy such as chordae tendineae and associated leaflets.

Reference is now made to FIGS. 5A and 5B, which are schematic illustrations of sectioned views of an anterior aspect of an exemplary heart (500, 550) showing an embodiment of a prosthetic heart valve device (mitral position 535, tricuspid position 555) implanted within both the mitral and tricuspid positions, in accordance with some applications of the invention. Specifically, FIG. 5A schematically illustrates an exemplary heart 500 that has been sectioned along a plane that bisects the pulmonary trunk 501, right atrium 502, left atrium 503, right ventricle 510 and left ventricle 505 in order to reveal the internal features and details of the chambers of the heart (right atrium 405, left atrium 445, right ventricle 420, and left ventricle 425) in relation to the design features of an exemplary embodiment of a prosthetic heart valve device 535 that has been designed for implantation within the mitral position. An exemplary embodiment of a prosthetic heart valve device 535 may be designed so as to have a minimized profile extending into both the inflow (left atrium 445 or right atrium 405) and outflow (right ventricle 420 or left ventricle 425) regions, in order to prevent ventricular outflow tract obstruction (left ventricular outflow tract 512, right ventricular outflow tract 520) and reduced ejection fraction in the case of outflow region obstruction, and blood flow disturbance and stasis formation in the case of inflow region obstruction. An exemplary embodiment of a prosthetic heart valve device 535 may also take advantage of native anatomy such as the anterior and posterior regions (545 and 540, respectively) of the mitral annulus (514, FIG. 5B), and use radial outward force to assist in device anchoring and also by having load bearing surfaces that may rest adjacent to both the floor of an atrium (left, 445) and the ceiling of a ventricle (left, 425), effectively clamping onto the native annulus and preventing device migration towards either the left atrium 445 or left ventricle 425. These features will be further described, below.

Similarly to FIG. 5A, FIG. 5B schematically illustrates an exemplary heart 550 that has been sectioned along a plane that bisects the pulmonary trunk 501, right atrium 502, left atrium 503, right ventricle 510 and left ventricle 505 in order to reveal the internal features and details of the chambers of the heart (right atrium 405, left atrium 445, right ventricle 420, and left ventricle 425) in relation to the design features of an exemplary embodiment of a prosthetic heart valve device 555 that has been designed for implantation within the tricuspid position, in accordance with some applications of the invention. This embodiment of an exemplary prosthetic heart valve device 555 may provide the same advantages as those found in the device previously described and designed for the mitral position. For example, an exemplary embodiment of a prosthetic heart valve device 555 may also take advantage of native anatomy such as the anterior, septal and posterior regions (565 and 560, respectively) of the tricuspid annulus (513, FIG. 5A), and use radial outward force to assist in device anchoring and also by having load bearing surfaces that may rest adjacent to both the floor of an atrium (right, 405) and the ceiling of a ventricle (right, 420), effectively clamping onto the native annulus and preventing device migration towards either the right atrium 405 or right ventricle 420.

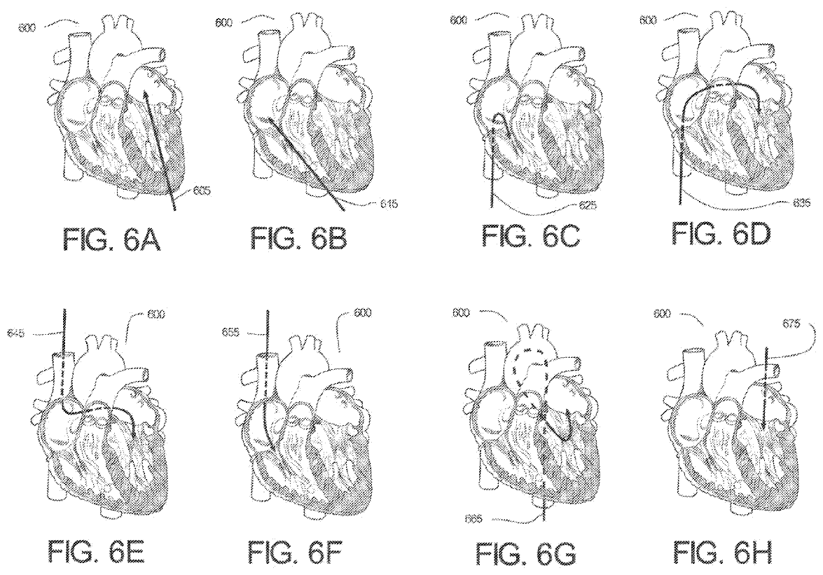

Reference is now made to FIGS. 6A-6H, which are schematic illustrations of sectioned views of an anterior aspect of an exemplary heart 600, showing the various percutaneous delivery pathways for an exemplary prosthetic heart valve device, in accordance with some applications of the invention. FIG. 6A illustrates the percutaneous pathway corresponding to transapical implantation within the mitral position, represented by directional arrow 605. FIG. 6B illustrates the percutaneous pathway corresponding to transapical implantation within the tricuspid position, represented by directional arrow 615. FIG. 6C illustrates the percutaneous pathway corresponding to transfemoral venous implantation within the tricuspid position, represented by directional arrow 625. FIG. 6D illustrates the percutaneous pathway corresponding to transfemoral venous/transseptal implantation within the mitral position, represented by directional arrow 635. FIG. 6E illustrates the percutaneous pathway corresponding to transsubclavian implantation within the mitral position, represented by directional arrow 645. FIG. 6F illustrates the percutaneous pathway corresponding to transsubclavian implantation within the tricuspid position, represented by directional arrow 655. FIG. 6G illustrates the percutaneous pathway corresponding to transaortic implantation within the mitral position, represented by directional arrow 665. FIG. 6H illustrates the percutaneous pathway corresponding to transatrial implantation within the mitral position, represented by directional arrow 675. While certain of the embodiments of exemplary prosthetic heart valve devices described herein are described in connection with a specific percutaneous delivery approach, it should be understood that these embodiments may be used for other percutaneous delivery approaches. Moreover, it should be understood that certain of the features described in connection with some embodiments can be incorporated with other embodiments, including those which are described in connection with different percutaneous delivery approaches, in accordance with some applications of the invention.

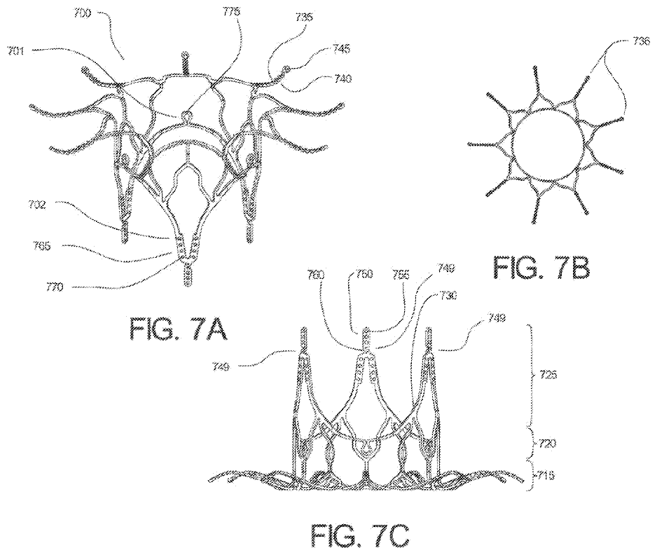

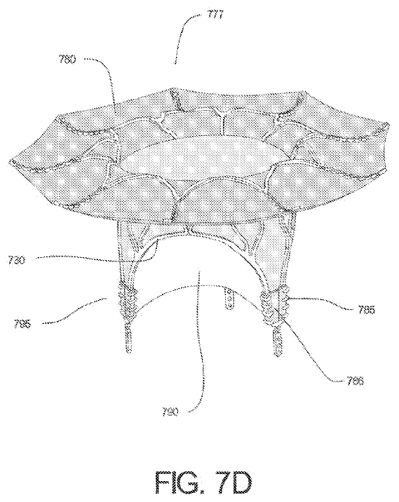

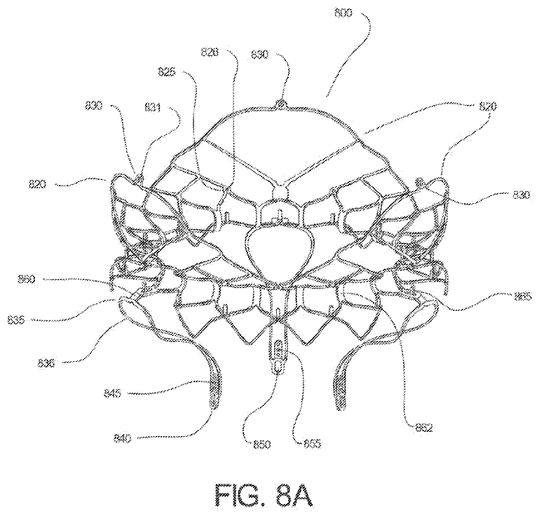

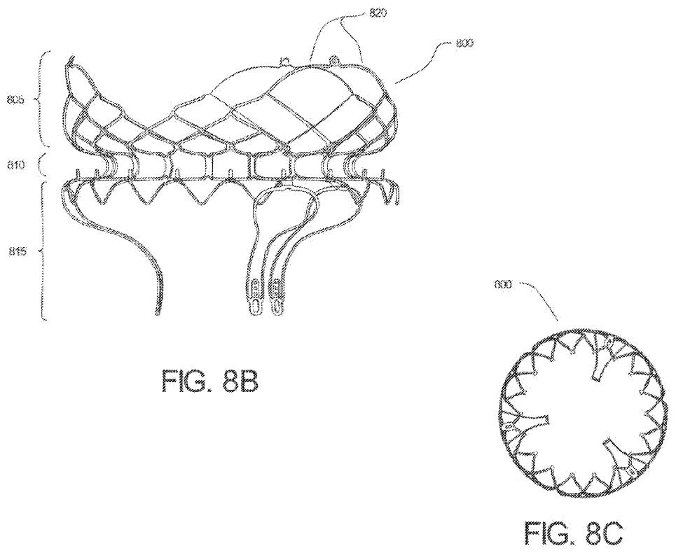

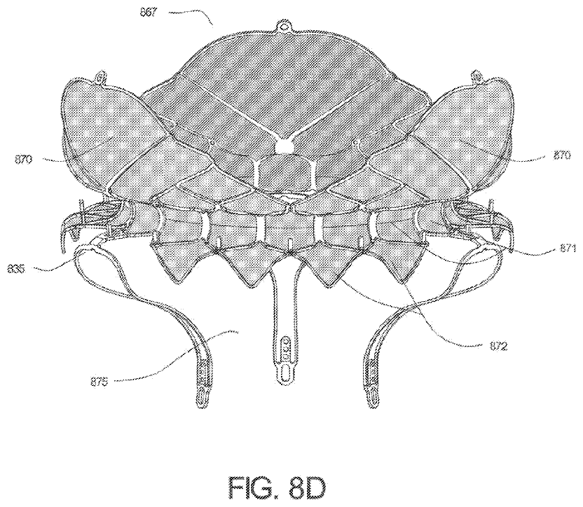

Reference is now made to FIGS. 7A-7D, which are schematic illustrations describing an embodiment of an exemplary self expanding valve frame 700 configured to mate with a differentially deformable anchoring structure (800, FIG. 8A), in accordance with some applications of the invention. Specifically, FIG. 7A illustrates a perspective view of an embodiment of an exemplary self expanding valve frame 700 that may be generally cylindrical in shape, having both an area of blood inflow 701, and an area of blood outflow 702 opposite the area of blood inflow 701, said areas generally describing the direction of which blood may flow through the device, during normal operation. The embodiment of an exemplary self expanding valve frame 700 described in FIG. 7A may be generally comprised of any alloy having super-elastic and shape-memory characteristics, such as Nitinol or any other super-elastic, shape-memorizing metallic or otherwise alloys, polymers, or compositions of material that may behave accordingly to a self expandable characteristic. Generally, an embodiment of an exemplary self expanding valve frame 700 may have a valve frame inflow region (715, FIG. 7C) adjacent to an area of blood inflow 701 and configured to provide features that prevent paravalvular leakage, as well as features that allow for mated connection between the exemplary self expanding valve frame 700 and an exemplary differentially deformable anchoring structure (800, FIG. 8A), adjacent to the valve frame inflow region (715, FIG. 7C). The features that allow for mated connection between the exemplary self expanding valve frame 700 and an exemplary differentially deformable anchoring structure (800, FIG. 8A) may further comprise a plurality (736, FIG. 7B) of elongate inflow region connection members 735 that are configured to flex and bend, allowing for structural distortion and absorption of force while still providing reliable and durable support between members. The inflow region connection member 735 may be further configured to include flexure geometry 740 that allows for said structural distortion and force absorption. The inflow region connection member 735 may also be configured to provide inflow region connection elements 745 which act as location features for a connectable mate between the inflow region connection member 735, and a corresponding atrial connection element (825, FIG. 8A) that is located on an embodiment of an exemplary differentially deformable anchoring structure (800, FIG. 8A). The features that allow for prevention of paravalvular leakage around the exemplary self expanding valve frame 700 may include a valve sealing cover (780, FIG. 7D) that may be comprised of fabrics such as polyester, nylon, PTFE, ePTFE, treated pericardial tissues, polymer fabrics, or any other material suitable for the construction of durable prosthetic heart valve devices, and that is configured to extend from the valve frame inflow region (715, FIG. 7C) to a valve frame outflow region (725, FIG. 7C, described below). Further, an embodiment of an exemplary self expanding valve frame 700 may also have a valve frame annular region (720, FIG. 7C) adjacent to and between both a valve frame inflow region (715, FIG. 7C) and a valve frame outflow region (725, FIG. 7C, described below) and configured to provide location for the connection of sutures and fabrics such as polyester, nylon, PTFE, ePTFE, treated pericardial tissues, polymer materials, or any other material suitable for the construction of durable prosthetic heart valve devices. The features that allow for the provision of location for the connection of sutures and fabrics to the exemplary self expanding valve frame 700 at the valve frame annular region (720, FIG. 7C) may include a leaflet attachment rail 730 to which sutures and fabrics may be attached, as well as a leaflet attachment rail flexure geometry (775, FIG. 7C) which may also accept sutures and fabrics, and further provide flexibility for aiding in the crimping process, prior to loading of the device into an exemplary delivery system (not shown) for percutaneous or otherwise implantation. Further still, an embodiment of an exemplary self expanding valve frame 700 may also have a valve frame outflow region (725, FIG. 7C) adjacent to and in a downstream direction from a valve annular region (720, FIG. 7C) and configured to provide features that allow for mated connection between the exemplary self expanding valve frame 700 and an exemplary differentially deformable anchoring structure (800, FIG. 8A), adjacent to the valve frame outflow region (725, FIG. 7C). The features that allow for mated connection between the exemplary self expanding valve frame 700 and an exemplary differentially deformable anchoring structure (800, FIG. 8A) at the valve frame outflow region (725, FIG. 7C) may include a plurality (749, FIG. 7C) of elongate outflow region connection members (750, FIG. 7C), extending from and adjacent to both the leaflet attachment rails (730, FIG. 7C), and the valve commissure attachment regions (765, FIG. 7A) that are configured to support the attachment of a plurality of leaflets (790, FIG. 7D), by way of sutures and commissural leaflet coupling elements (770, FIG. 7A). Each outflow region connection member (750, FIG. 7C) may further comprise a series of outflow region connection elements (755, FIG. 7C) which act as location features for a connectable mate between the outflow region connection member (750, FIG. 7C), and a corresponding ventricular region connection element (845, FIG. 8A) that is adjacent to a ventricular conformance structure support strut (836, FIG. 8A) that is located on an embodiment of an exemplary differentially deformable anchoring structure (800, FIG. 8A). Each outflow region connection member (750, FIG. 7C) may further comprise a flexure geometry (760, FIG. 7C) that is configured to flex and bend, allowing for structural distortion and absorption of force while still providing reliable and durable support between members.

With reference to FIG. 7D, a schematic illustration of a front view of an exemplary embodiment of a self expanding valve frame 777, which includes tissue leaflets and fabric coverings (valve sealing cover) for preventing paravalvular leakage 780 is depicted, in accordance with some applications of the invention. The embodiment of a self expanding valve frame 777 of FIG. 7D includes a leaflet attachment rail 730, which provides location for a plurality of leaflets 790. The leaflets may be comprised of a chemically treated and biologically compatible pericardial tissue material, or a biocompatible polymeric material, or any other material that is biocompatible and suitable for creation of prosthetic heart valve leaflet construction. Each leaflet 790 extends between a valve commissure 795 that is adjacent to and between the extents of each leaflet attachment rail 730, the valve commissure 795 being further comprised of commissure coverings 786, and attachment sutures 785.

Reference is now made to FIGS. 8A-8D, which are schematic illustrations that describe various views of an embodiment of an exemplary differentially deformable anchoring structure 800, in accordance with some applications of the invention. The embodiment of an exemplary differentially deformable anchoring structure 800 depicted in FIGS. 8A-8B may be comprised of an anchor atrial region 805, generally comprised of a plurality of elongate struts that collectively define diamond shaped cell structures, and that generally have a first stiffness. The atrial region 805 may be configured to conform to an atrial surface of a native antrioventricular valve of a heart (see FIGS. 5A-5B), and provide resistance to migration from an atrium of an atrioventricular valve towards a corresponding ventricle of a heart. The atrial region 805 may further comprise a plurality of atrial release members 830, each adjacent to and extending from an atrial conformance structure 820 that is configured to also provide a smooth surface upon which an exemplary delivery system catheter (not shown) may be drawn to capture and sheath the prosthetic heart valve device of this disclosure. The atrial release member 830 may be further configured to include atrial release member geometry 831 that allows for a releasable connection between the differentially deformable anchoring structure 800, and an exemplary delivery system (not shown). An additional feature of the exemplary differentially deformable anchoring structure 800 may include atrial region connection elements 825 having atrial connection element geometry 826 that is configured to connectedly mate to inflow region connection elements 745 of an exemplary self expanding heart valve frame (700, FIG. 7A).

The embodiment of an exemplary differentially deformable anchoring structure 800 schematically illustrated in FIGS. 8A-8B may further comprise an anchor annular region 810, generally comprised of a plurality of elongate and broad annular region clasping struts 862 that collectively define a ring-like circumferential structure, traversing the circumference of the exemplary differentially deformable anchoring structure 800 of this embodiment, and that generally have a second stiffness. The annular region 810 may be configured to conform to an annulus of a native antrioventricular valve of a heart (see FIGS. 5A-5B) and provide resistance to migration away from the aforementioned annulus by way of radial expansion force. Additionally, the embodiment of an exemplary differentially deformable anchoring structure 800 depicted in FIGS. 8A-8B may further comprise an anchor ventricular region 815, generally having a third stiffness and generally being comprised of a plurality of elongate and broad ventricular conformance structures 835 that comprise a heel 860 for abutting against the ceiling of a native ventricle (see FIGS. 5A-5B), and a plurality of elongate ventricular conformance structure support struts 836 that terminate at a ventricular release member 840; the ventricular release member 840 having a ventricular release member geometry 850 that is configured to releasably connect the differentially deformable anchoring structure 800, to an exemplary delivery system (not shown). Each ventricular conformance structure 835 may further comprise a plurality of ventricular region connection elements 845, each having a ventricular region connection element geometry 855 that provides for mated connection to outflow region connection elements 755 of an exemplary self expanding heart valve frame (700, FIG. 7A). The heel of the ventricular region conformance structure 860 may further comprise annular anchoring elements 865, which are configured to pierce annular tissue and enhance the anchoring force of the differentially deformable anchoring structure 800. Finally, the ventricular region 815 may be configured to conform to a ventricular wall and annulus of a native antrioventricular valve of a heart (see FIGS. 5A-5B), and provide resistance to migration away from the aforementioned annulus and towards an atrium, by way of abutment of the heel 860 against the ceiling of a ventricle, in a location adjacent to a subvalvular surface of said native annulus. The first stiffness of the atrial region 805, the second stiffness of the annular region 810, and the third stiffness of the ventricular region 815 may be related in such a manner as to provide an appropriate combination of optimized stiffnesses for avoiding device migration, as well as conformance to native structures of a native heart. The stiffnesses may generally be equal; Alternatively, the first stiffness may generally be more or less stiff than one or both of the second and third stiffnesses. Further, the second stiffness may generally be more or less stiff than one or both of the first and third stiffnesses. Finally, the third stiffness may generally be more or less stiff than one or both of the first and second stiffnesses.

Reference is now made to FIG. 8D, which is a schematic illustration of an embodiment of a differentially deformable anchoring structure having fabric coverings 867, in accordance with some applications of the invention. The anchoring structure having fabric coverings 867 may be comprised of the aforementioned differentially deformable anchoring structure 800, in addition to an anchor sealing cover 870 configured to prevent paravalvular leakage and comprised of fabrics such as polyester, nylon, PTFE, ePTFE, treated pericardial tissues, polymer fabrics, or any other material suitable for the construction of durable prosthetic heart valve devices. The anchor sealing cover 870 may further comprise an annular region sealing cover 871 and annular region sealing cover diamonds 872, in order to provide maximized fabric surface area, and thus maximized resistance to paravalvular leakage. Finally, a triad of ventricular region outflow openings 875 may each be formed by the boundary of an annular region sealing cover 871, in conjunction with a plurality of ventricular conformance structures 835, and configured to maximize the space available beneath the described embodiment of a prosthetic heart valve device, and the ventricular outflow tract in which the device will be implanted (see FIGS. 5A-5B), in order to reduce the occurrence of ventricular outflow tract obstruction.

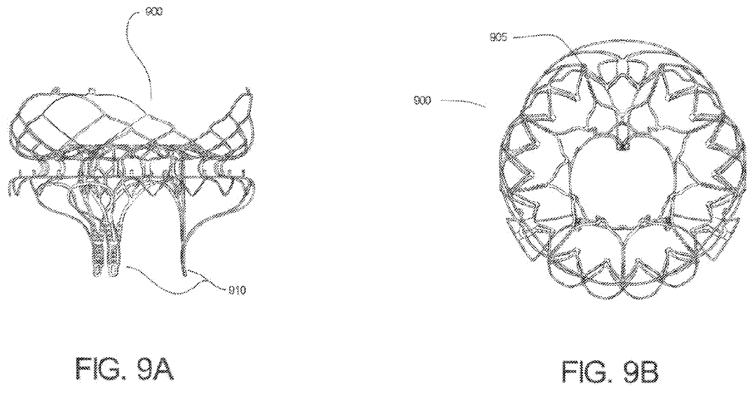

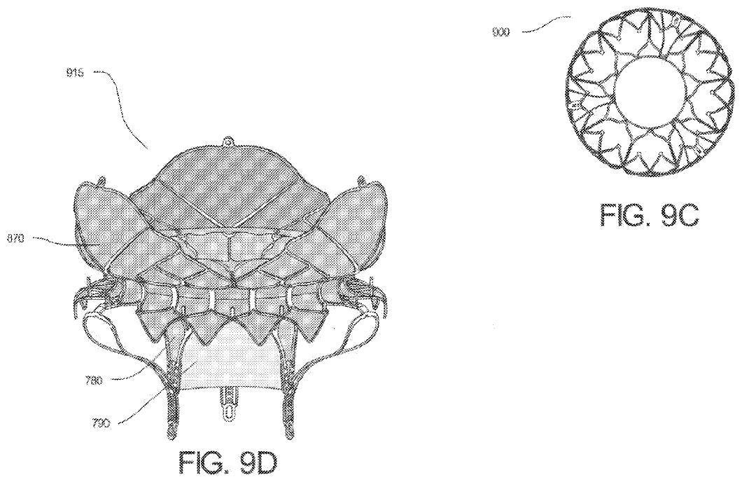

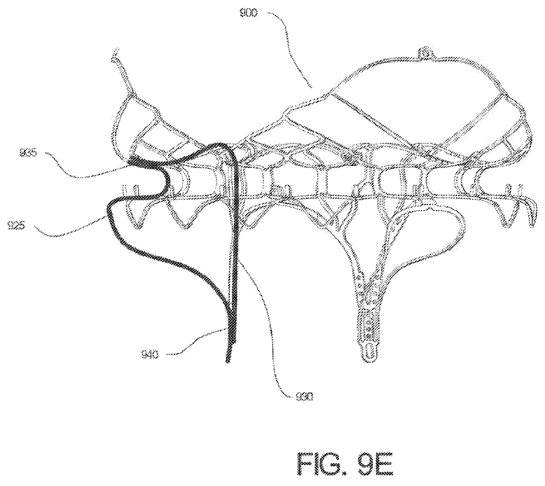

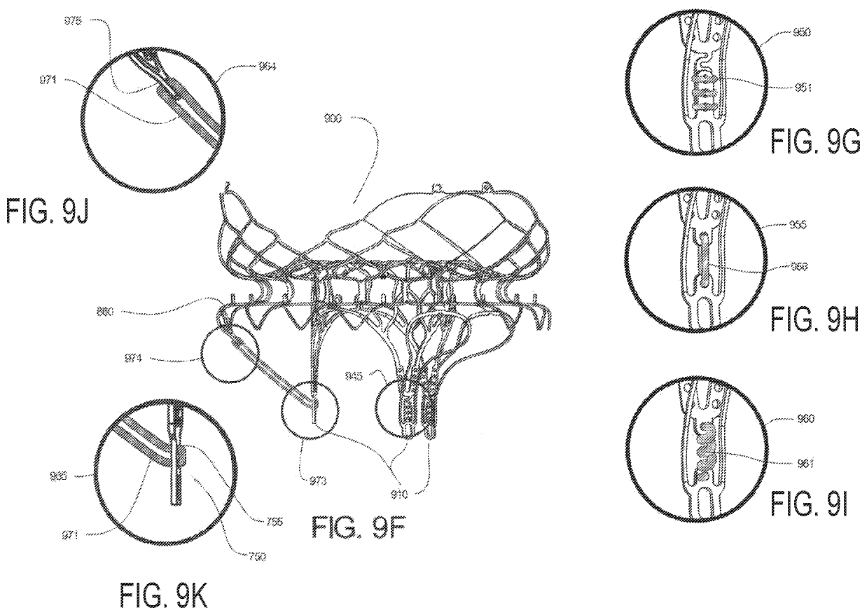

Reference is made to FIGS. 9A-9F, which are schematic illustrations depicting various views of an embodiment of an exemplary prosthetic heart valve device 900, in accordance with some applications of the invention. Specifically, FIG. 9A illustrates a front view of an embodiment of an exemplary prosthetic heart valve device 900, while FIG. 9B illustrates a perspective view of said prosthetic heart valve device 900, and FIG. 9C illustrates a perspective overhead (inflow) view of said prosthetic heart valve device 900, while FIG. 9D illustrates a front view of said prosthetic heart valve device with coverings 915. Finally, FIG. 9E illustrates a cross-sectional profile view of said exemplary prosthetic heart valve device 900. Turning to FIG. 9A, a mated connection at the outflow end 910 between an embodiment of an exemplary self expanding heart valve frame (700, FIG. 7A), and an exemplary embodiment of a differentially deformable anchoring structure (800, FIG. 8A) can be seen. In FIG. 9B, a mated connection at the inflow end 905 between an embodiment of an exemplary self expanding heart valve frame (700, FIG. 7A), and an exemplary embodiment of a differentially deformable anchoring structure (800, FIG. 8A) can similarly be seen. With reference to FIG. 9D, an exemplary embodiment of a prosthetic heart valve device with coverings 915 is schematically illustrated, with valve sealing cover 780, leaflets 790, and anchor sealing cover 870 in view, in accordance with some applications of the invention. Referring now to FIG. 9E, a cross-sectional view of an exemplary embodiment of a prosthetic heart valve device 900 is schematically illustrated, in accordance with some applications of the invention. A highlighted curve depicting an anchor cross-section 925 is shown adjacent to a highlighted curve depicting a valve frame cross-section 930. An embodiment of a prosthetic heart valve device 900 may be designed such that the entire length of the highlighted curve depicting an anchor cross-section 925 is of an equivalent length to the entire length of a highlighted curve depicting a valve frame cross-section 930, such that when each curve is connected as in the assembled device with coverings 915 (connection at inflow 935, and connection at outflow 940) illustrated in FIG. 9D, the heart valve frame (700, FIG. 7A) and differentially deformable anchor structure (800, FIG. 8A) will collapse together and uniformly, when placed under tension applied at both the inflow and outflow ends, for example when being loaded into an exemplary embodiment of a delivery system catheter (described further below).

Finally, FIG. 9F depicts various alternative embodiments of connection configurations for connecting the ventricular region connection element geometry (855, FIG. 8A) of the anchor to the outflow region connection elements 755 of the valve frame. Specifically, detail section circles 945, 973, and 974 illustrate five reference lines (946, 947, 948, 963, 962) leading to respective enlarged section circles (950, 955, 960, 965, 964) that each describe an alternative embodiment of a connection configuration. Reference line 946 leads from a first detailed section circle 945 to enlarged section circle 950, and depicts an embodiment of a connection configuration comprising a suture-like or filament type material 951 that has been interwoven between the ventricular region connection element geometry (855, FIG. 8A) of the anchor and the outflow region connection elements 755 of the valve frame, that is configured to create a rigid connection. The suture-like or filament type material 951 can comprise an elastic or flexible textile or polymer. The suture-like or filament type material 951 can also comprise a flexible or elastic metallic alloy. The suture-like or filament type material 951 can also comprise a rigid and un-flexible material, polymer, textile, or alloy. Reference line 947 leads from a first detailed section circle 945 to enlarged section circle 955, and depicts an embodiment of a connection configuration comprising a suture-like or filament type material 956 that has been connected between the ventricular region connection element geometry (855, FIG. 8A) of the anchor and the outflow region connection elements 755 of the valve frame. The suture-like or filament type material 956 can be configured to provide for a connection that allows for some displacement between the ventricular region connection element geometry (855, FIG. 8A) of the anchor and the outflow region connection elements 755 of the valve frame. The suture-like or filament type material 956 can comprise an elastic or flexible textile or polymer. The suture-like or filament type material 956 can also comprise a flexible or elastic metallic alloy. The suture-like or filament type material 956 can also comprise a rigid and un-flexible material, polymer, textile, or alloy. Reference line 948 leads from a first detailed section circle 945 to enlarged section circle 960, and depicts an embodiment of a connection configuration comprising a coil-like material 961 that has been connected between the ventricular region connection element geometry (855, FIG. 8A) of the anchor and the outflow region connection elements 755 of the valve frame. The coil-like material 961 can be configured to provide for a connection that allows for maximum displacement between the ventricular region connection element geometry (855, FIG. 8A) of the anchor and the outflow region connection elements 755 of the valve frame. The coil-like material 961 can comprise an elastic or flexible textile or polymer. The coil-like material 961 can also comprise a flexible or elastic metallic alloy. The coil-like material 961 can also comprise a rigid and un-flexible material, polymer, textile, or alloy.

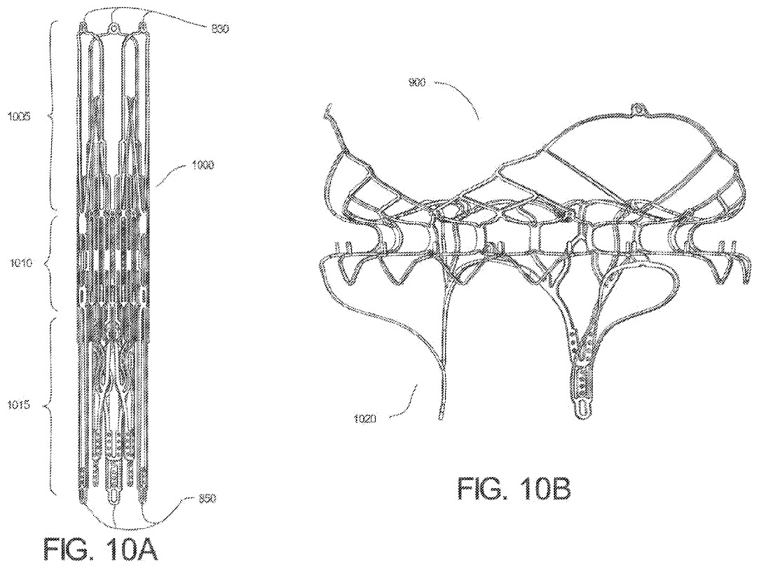

Reference line 962 leads from a second detailed section circle 974 to enlarged section circle 964, and depicts an alternative embodiment of a connection configuration comprising a suture-like material 971 that has been directly connected between an alternative embodiment of ventricular region connection element geometry 975 of the anchor (adjacent to and extending from the heel 860), and the outflow region connection elements 755 of the valve frame. In this particular embodiment, one or more ventricular conformance structure support struts 836 can be replaced by direct-connections with suture-like material 971, enabling a tensile connection, or a rigid connection, or a connection that may absorb some displacement between connected components. The connection configuration depicted in this specific alternative embodiment may be realized at one or more, or none of the valve commissure regions (795, FIG. 7D). The connection configuration depicted in this specific alternative embodiment may be designed so as to isolate any affected valve commissure region (795, FIG. 7D) from annular deformations induced upon the anchor. The connection configuration depicted in this specific alternative embodiment may further be designed so as to reduce the overall crimped height (vertical distance between elements 830 and 850 as depicted in FIG. 10A) of the device.

Reference line 963 leads from a third detailed section circle 973 to enlarged section circle 965, and depicts a view of the opposite end (focusing on an outflow region connection member 750) of the alternative embodiment described above of a connection configuration comprising a suture-like material 971 that has been directly connected between an alternative embodiment of ventricular region connection element geometry 975 of the anchor (adjacent to and extending from the heel 860), and the outflow region connection elements 755 of the valve frame.

Reference is now made to FIGS. 10A and 10B, which are schematic illustrations of front views of an exemplary embodiment of a prosthetic heart valve device 900 in both the crimped configuration 1000 and expanded configuration 1020, in accordance with some applications of the invention. Specifically, FIG. 10A illustrates the crimped configuration 1000 which would arise when the prosthetic heart valve device 900 has been crimped and loaded into an exemplary embodiment of a delivery system catheter (described further below) through radial compression or axial tension. The atrial region 1005, annular region 1010 and ventricular region 1015 when in the crimped configuration 1000 can also be seen. Similarly, FIG. 10B illustrates the expanded configuration 1020 which would arise when the prosthetic heart valve device 900 has been fully released and implanted within a native atrioventricular valve.

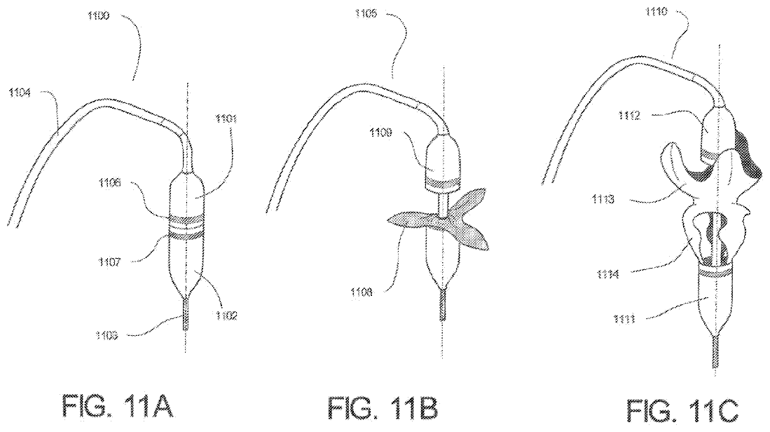

Reference is made to FIGS. 11A-11C which are schematic illustrations depicting a sequence showing a typical deployment process of an exemplary embodiment of a prosthetic heart valve device 900 being deployed by an exemplary embodiment of a delivery system 1100, in accordance with some applications of the invention. FIG. 11A illustrates a pre-deployment configuration of an exemplary section of catheter 1104 that is adjacent to a proximal capsule portion 1101 and a distal capsule portion 1102. The proximal capsule portion 1101 may have a proximal marker band 1106, and the distal capsule portion 1102 may have a distal marker band 1107 in order to assist in imaging guidance for implantation procedures. The exemplary embodiment of a delivery system 1100 may be configured to travel upon a guidewire 1103 in order to track the device into position during an implantation procedure. FIG. 11B illustrates a mid-deployment configuration of an exemplary section of catheter 1104 of an exemplary embodiment of a delivery system 1105 which shows that the proximal capsule portion 1109 has been translated away from the distal capsule portion 1102, revealing an atrial portion of an exemplary embodiment of a prosthetic heart valve device 1108. FIG. 11C illustrates a full deployment configuration of an exemplary section of catheter 1104 of an exemplary embodiment of a delivery system 1110 which shows that both the proximal capsule portion 1112 and distal capsule portion 1111 have been fully translated away from each other, fully revealing both atrial 1113 and ventricular 1114 portions of an exemplary embodiment of a prosthetic heart valve device 900, in accordance with some applications of the invention. It should be understood that in this exemplary embodiment of a prosthetic heart valve device 900, both the atrial 1113 and ventricular 1114 portions have not yet been fully released.

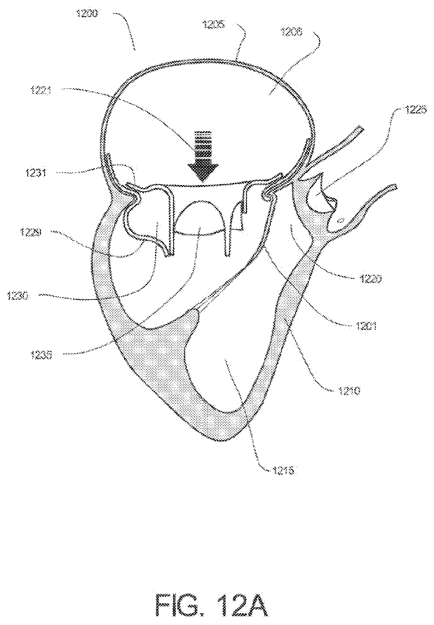

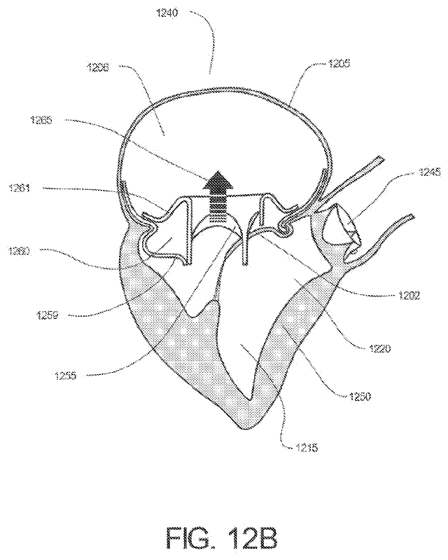

Reference is made to FIGS. 12A-12B, which are schematic illustrations depicting a sequence showing the transition between the cardiac cycle phases of diastole and systole with particular reference to a cross-sectioned heart (diastole 1200, systole 1240, FIGS. 12A, 12B respectively) and an exemplary prosthetic heart valve device (diastolic embodiment 1230, systolic embodiment 1260, FIGS. 12A, 12B respectively) implanted in-situ, in accordance with some applications of the invention. Specifically, FIG. 12A illustrates an exemplary diastolic embodiment of a prosthetic heart valve device 1230 that has been implanted in the mitral position. The open leaflets 1235 of the exemplary diastolic embodiment of a prosthetic heart valve device 1230 are acting in response to the inflow of blood from the left atrium 1206 (cross-section 1205) and towards the left ventricle 1215 during diastolic ventricular filling. Similarly, the closed leaflets of an exemplary aortic valve 1225 are also acting in response to said diastolic ventricular filling. Directly beneath the closed leaflets of the exemplary aortic valve 1225 can be seen the left ventricular outflow tract 1220 and the left ventricular wall in cross-section 1210, in an expanded state. Directly above the exemplary diastolic embodiment of a prosthetic heart valve device 1230 can be seen an arrow 1221 that corresponds to the attitude of the exemplary diastolic embodiment of a heart valve frame 1231, the attitude being positionally un-displaced with respect to the native annulus in which an exemplary diastolic embodiment of a differentially deformable anchoring structure 1229 sits. Adjacent to the exemplary diastolic embodiment of the prosthetic heart valve device 1230 can be seen a native anterior leaflet 1201, depicted in a free, open, and unfettered position. It shall be understood that the exemplary embodiments of native anatomy and prosthetic heart valve devices depicted in FIG. 12A may also be realized in such a manner with respect to the anatomy of an alternative atrioventricular valve such as a tricuspid valve, with its corresponding native tricuspid anatomy.

Reference is now made to FIG. 12B, which is a schematic illustration of an exemplary systolic embodiment of a prosthetic heart valve device 1260 that has been implanted in the mitral position, with specific reference now to a cross-sectioned heart in systole 1240, in accordance with some applications of the invention. The closed leaflets 1255 of the exemplary systolic embodiment of a prosthetic heart valve device 1260 are acting in response to the pressurization of the left ventricle 1215, and hence enable the outflow of blood from the left ventricle 1215 (cross-section 1250) to the left ventricular outflow tract 1220, and out through the open aortic valve 1245 during systolic ventricular contraction. The unfettered native anterior leaflet 1202 can be seen in the closed position, abutted against an anterior aspect of the exemplary systolic embodiment of a prosthetic heart valve device 1260. Directly above the exemplary systolic embodiment of a prosthetic heart valve device 1260 can be seen an arrow 1265 that corresponds to the attitude of the exemplary systolic embodiment of the heart valve frame 1261, the attitude being positionally displaced in an atrial direction with respect to the native annulus in which an exemplary systolic embodiment of the differentially deformable anchoring structure 1259 sits. It shall be understood that the exemplary embodiments of native anatomy and prosthetic heart valve devices depicted in FIG. 12B may also be realized in such a manner with respect to the anatomy of an alternative atrioventricular valve such as a tricuspid valve, with its corresponding native tricuspid anatomy.

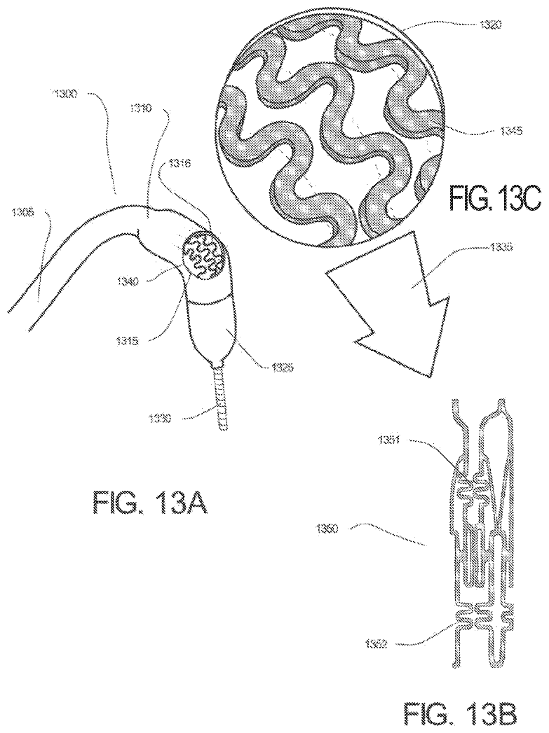

Reference is made to FIG. 13A, which is a schematic illustration describing a perspective view of a detailed section 1315 of an embodiment of an exemplary prosthetic heart valve device 1340 loaded into an exemplary delivery system 1300, in accordance with some applications of the invention. An exemplary embodiment of a loaded delivery system in a bent configuration 1300 may include a proximal portion of a capsule 1310 located adjacent to a proximal neck 1305, and a distal portion of a capsule 1325 adjacent to the proximal portion 1310, wherein each capsule portion is configured to translate away from the opposite capsule portion during deployment. The exemplary embodiment of the loaded delivery system in a bent configuration 1300 may be configured to be railed into anatomical position over top of a guidewire 1330 that can be procedurally placed into position, prior to the introduction of the delivery system 1300 catheter. A broken-out section view window 1315 enables a partially revealed section 1340 of exemplary prosthetic heart valve device frame to be seen, showing an embodiment of flexure geometry 1316. The flexure geometry 1316 may be configured to allow specific portions of an exemplary embodiment of a heart valve device 1340 to be bent into specific orientations and bend radii, suitable for tracking through native anatomical vessels, veins and arteries and into position within a native atrioventricular valve. An enlarged view 1320 of broken out section window details the enlarged and partially revealed flexure geometry 1345. Turning now to FIG. 13B by following an arrow 1335 depicting the reference to FIG. 13B, a segment of exemplary prosthetic heart valve device frame flat pattern 1350 is schematically illustrated, in accordance with some applications of the invention. The exemplary prosthetic heart valve device frame flat pattern 1350 may include an exemplary embodiment of atrial region flexure elements 1351 configured to allow for specific bending of the prosthetic heart valve device at an atrial region, as well as an exemplary embodiment of ventricular region flexure elements 1352 configured to allow for specific bending of the prosthetic heart valve device at a ventricular region.

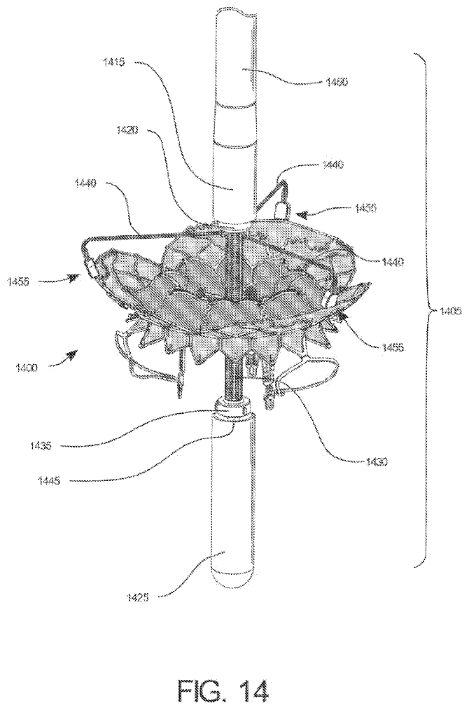

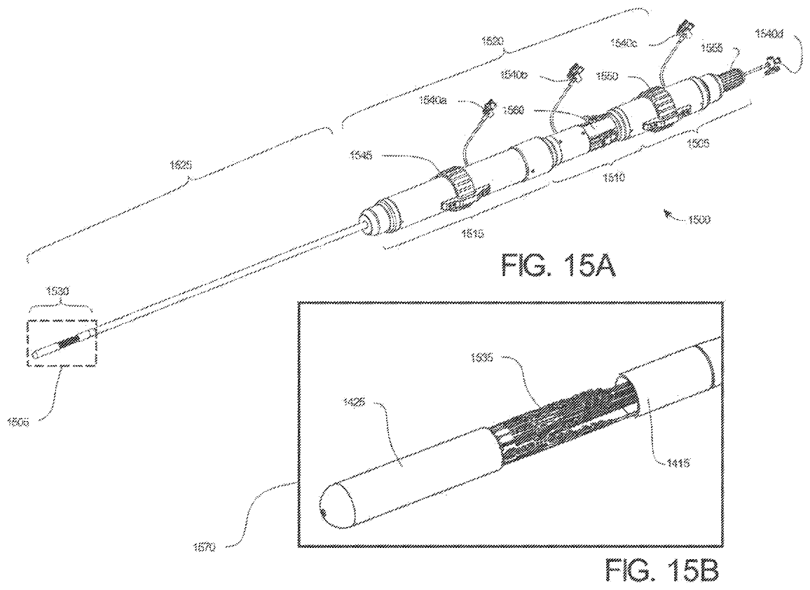

Reference is made to FIG. 14, which is a schematic illustration of a distal portion 1405 of an exemplary embodiment of a delivery system (1500, FIG. 15A), with an exemplary embodiment of a prosthetic heart valve device 1400 loaded in a partially deployed configuration for illustrative purposes. The prosthetic heart valve device 1400 is in accordance with some applications of the invention, as previously described. The exemplary delivery system (1500, FIG. 15A) can comprise an assembly of concentrically aligned and radially adjacent flexible catheters, including a first catheter 1420, a second catheter 1430 configured to extend at least partially through the first catheter 1420, a third catheter 1445 configured to extend at least partially through the second catheter 1430, and a fourth catheter 1450 configured to extend at least partially overtop of the first catheter 1420. The fourth catheter 1450 can have a proximal outer covering section 1415. The third catheter 1445 can have a distal outer covering section 1425. The second catheter 1430 can have a connection element 1435 for connecting to a portion of the exemplary prosthetic heart valve device 1400. The first catheter 1420 can house a plurality of tethers 1440, configured to matingly connect to a portion of the prosthetic heart valve device 1400 at an atrial region. The tethers may further comprise a plurality of tether connector apparatuses 1455 that may provide the means through which the tethers matingly connect to the prosthetic heart valve device, details of which shall be provided further below. Additional details about the aforementioned catheters are also provided, further below.

Reference is made to FIGS. 15A-B which are schematic illustrations of an exemplary delivery system 1500 loaded with a prosthetic heart valve device 1535 in a compressed delivery state, in accordance with some applications of the invention.

Delivery system 1500 is configured for intracardiac delivery of the compressed prosthetic heart valve device 1535 and comprises a handle portion 1520, and a catheter portion 1525 adjacent to and extending distally from the handle portion 1520.

Handle portion 1520 has a generally elongate shape and is generally cylindrical, having a proximal region 1505, a distal region 1515, and a mid region 1510 therebetween.

Catheter portion 1525 extends distally from the distal region 1515 of the handle portion 1520 and can comprise one or more flexible catheters, such as a first catheter 1420 and a second catheter 1430, which extends through first catheter 1420 such that a flexible distal portion of second catheter 1430 is disposed out of the distal end of first catheter 1420. The distal portion of the second catheter 1430 may further comprise a connection element 1435 configured for releasable attachment to at least a portion of the compressed prosthetic heart valve device 1535.

Catheter portion 1525 of delivery system 1500 further comprises a third catheter 1445 which extends through second catheter 1430 such that a distal outer covering section 1425 is disposed out of the distal end of second catheter 1430.

Catheter portion 1525 of delivery system 1500 further comprises a fourth catheter 1450 which covers a portion of the first catheter 1420 and comprises a proximal outer covering section 1415 that may extend over at least a portion of the compressed prosthetic heart valve device 1535.

Catheter portion 1525 of delivery system 1500 further comprises a retention region 1530, configured for retaining a compressed prosthetic heart valve device 1535 for delivery. For example, distal outer covering section 1425 of the third catheter 1445 and proximal outer covering section 1415 of the fourth catheter 1450 can act as constraining members, each radially constraining at least a portion of compressed prosthetic heart valve device 1535 in a compressed delivery state therewith, thereby retaining the compressed prosthetic heart valve device 1535.

Distal region 1515 of handle portion 1520 generally comprises a first thumbwheel 1545 that is in controllable communication with fourth catheter 1450 through a mechanical interaction internal to the distal region 1515 (described in further detail below). Actuation of the first thumbwheel 1545 can controllably translate the fourth catheter 1450 from a first position (proximal) to a second position (distal) further downstream than the first, and back. When in the second position (distal), the proximal outer covering section 1415 of the fourth catheter 1450 can be in a favorable position for constraining at least a portion of the compressed prosthetic heart valve device 1535. When in the first position (proximal), the proximal outer covering section 1415 of the fourth catheter 1450 can be in a favorable position for releasing at least a portion of the compressed prosthetic heart valve device 1535 from radial constraint.

Distal region 1515 of handle portion 1520 generally further comprises a saline flush port 1540a, which can facilitate removal of entrapped air from between concentrically adjacent catheters during device preparation, for example, removal of air from between the fourth catheter 1450 and the first catheter 1420 by allowing for the injection of sterile saline therebetween said catheters 1420 and 1450, thereby removing said entrapped air and preventing the introduction of air emboli to the bloodstream.

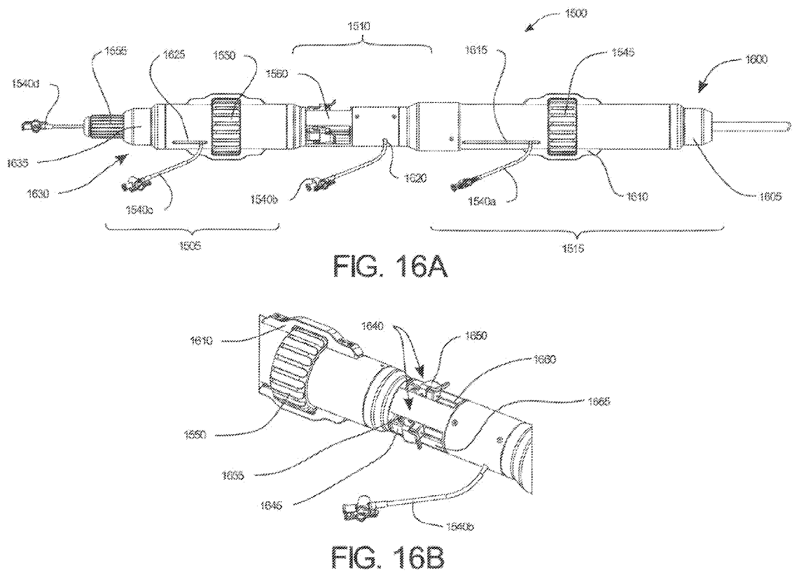

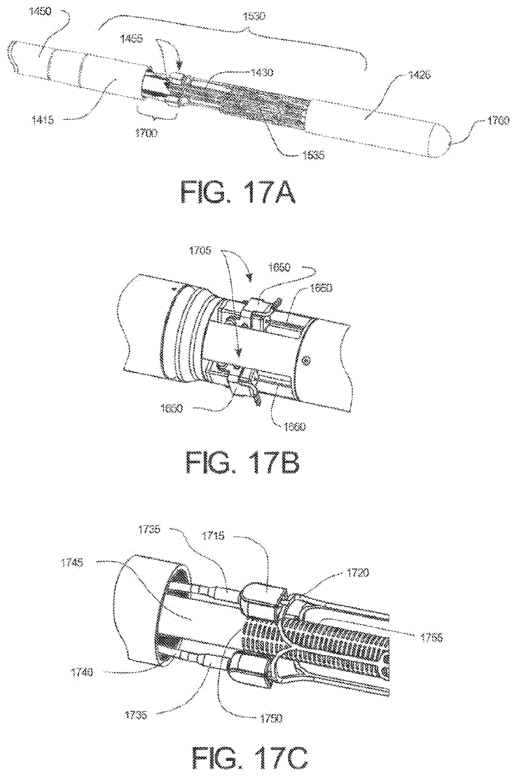

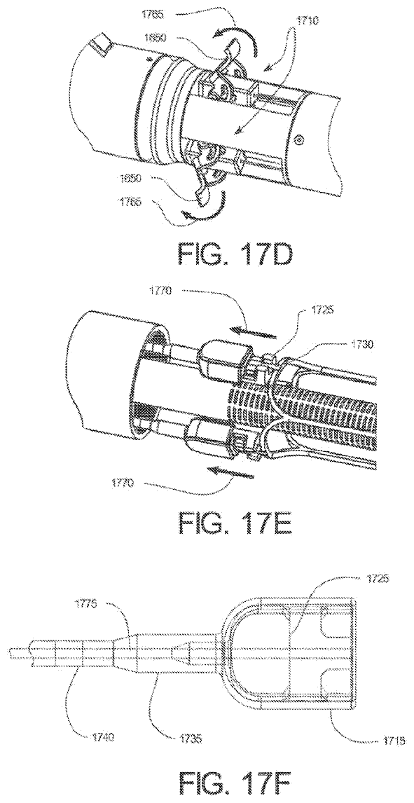

Mid region 1510 of handle portion 1520 generally comprises a saline flush port 1540b, and a tether shuttle assembly 1560, the details of which shall be provided further below, with reference to FIGS. 16A-B, and FIGS. 17A-E. The saline flush port 1540b of the mid region 1510 can facilitate removal of entrapped air from between concentrically adjacent catheters during device preparation, for example, removal of air from between first catheter 1420 and the second catheter 1430 by allowing for the injection of sterile saline therebetween said catheters 1420 and 1430, thereby removing said entrapped air and preventing the introduction of air emboli to the bloodstream. Mid region 1510 of handle portion 1520 can also comprise a location for the internal mechanical attachment of the first catheter 1420 to the handle portion 1520.

Proximal region 1505 of the handle portion 1520 generally comprises a second thumbwheel 1550 that is in controllable communication with second catheter 1430 through a mechanical interaction internal to the proximal region 1505 (described in further detail below). Actuation of the second thumbwheel 1550 can controllably translate the second catheter 1430 from a first position (proximal) to a second position (distal) further downstream than the first, and back. When in the second position (distal), the compressed prosthetic heart valve device 1535 can be in a more distally located position (for example, while within a ventricle of a heart) while loaded for delivery. When in the first position (proximal), the compressed prosthetic heart valve device 1535 can be in a more proximally located position while loaded for delivery.

Proximal region 1505 of the handle portion 1520 may further comprise a third thumbwheel 1555 that is in controllable communication with the third catheter 1445 through a mechanical interaction internal to the proximal region 1505 (described in further detail below). Actuation of the third thumbwheel 1555 can controllably translate the third catheter 1445 from a first position (proximal) to a second position (distal) further downstream than the first, and back. When in the first position (proximal), the distal outer covering section 1425 of the third catheter 1445 can be in a favorable position for constraining at least a portion of the compressed prosthetic heart valve device 1535. When in the second position (distal), the distal outer covering section 1425 of the third catheter 1445 can be in a favorable position for releasing at least a portion of the compressed prosthetic heart valve device 1535 from radial constraint.

Proximal region 1505 of handle portion 1520 generally further comprises a saline flush port 1540c, which can facilitate removal of entrapped air from between concentrically adjacent catheters during device preparation, for example, removal of air from between the second catheter 1430 and the third catheter 1445 by allowing for the injection of sterile saline therebetween said catheters 1430 and 1445, thereby removing said entrapped air and preventing the introduction of air emboli to the bloodstream. Proximal region 1505 of handle portion 1520 also further comprises a saline flush port 1540d, which can facilitate removal of entrapped air from within a guidewire lumen that runs from a first end of the third catheter 1445 to a second end, opposite the first by allowing for the injection of sterile saline therein, thereby removing said entrapped air and preventing the introduction of air emboli to the bloodstream.

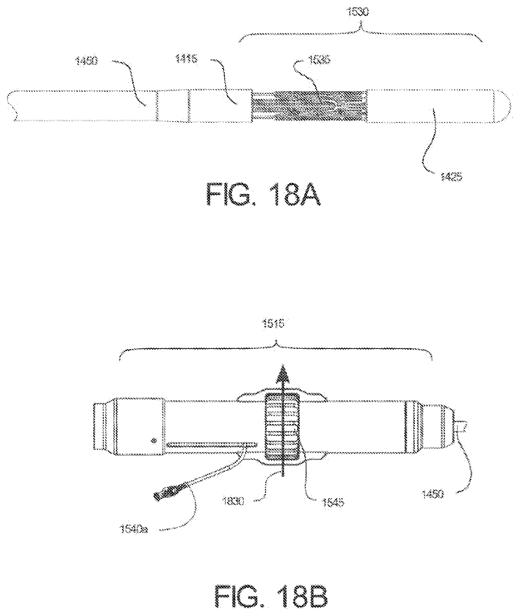

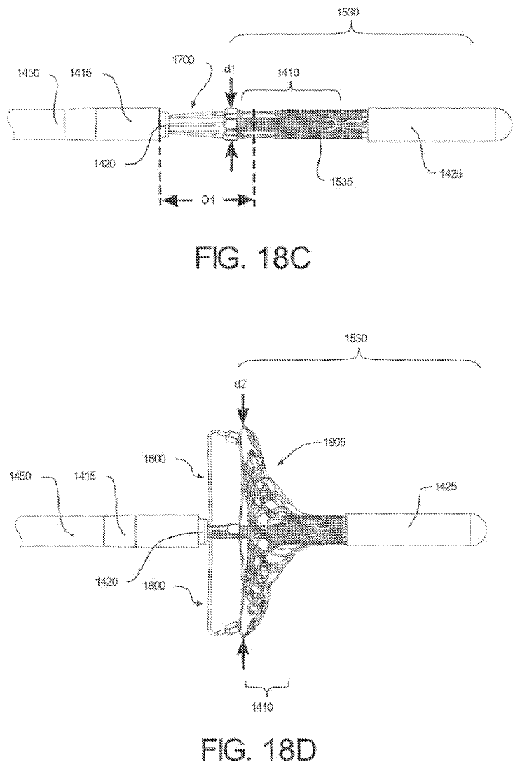

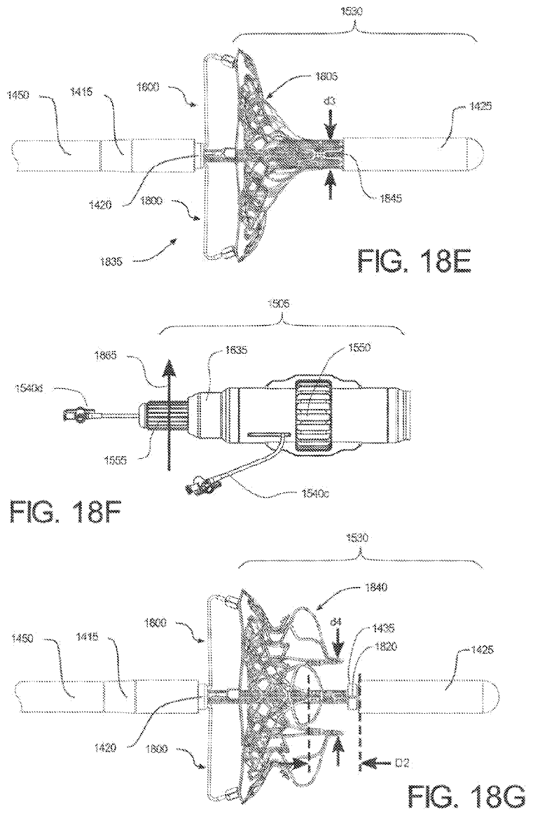

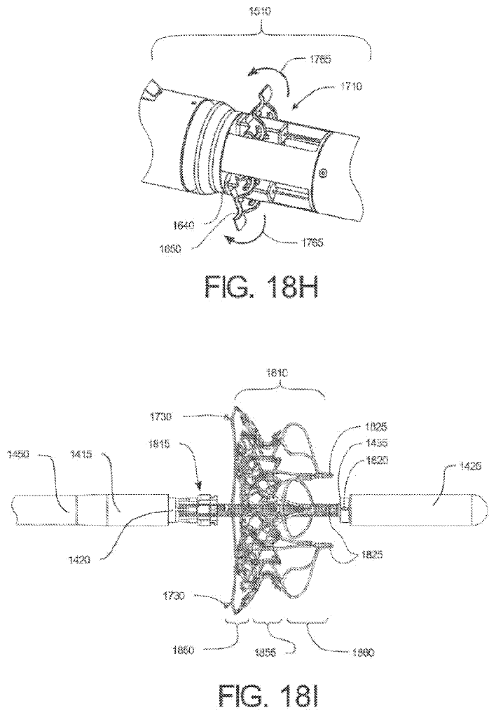

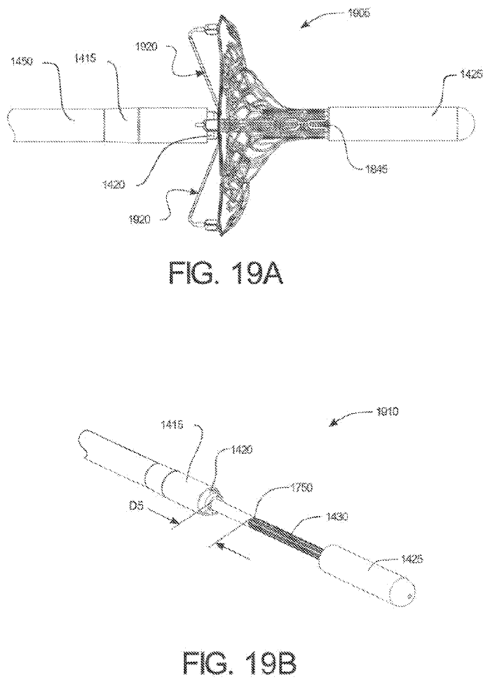

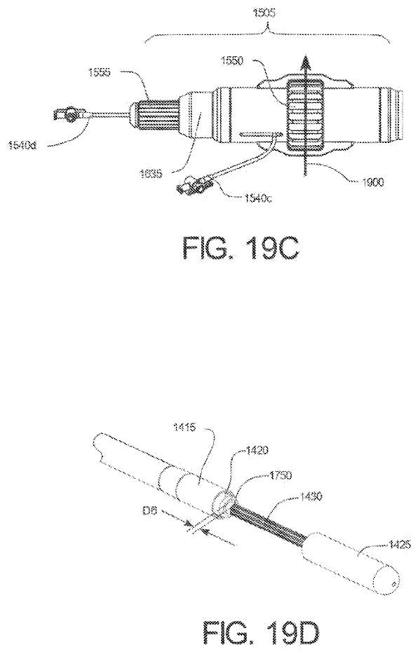

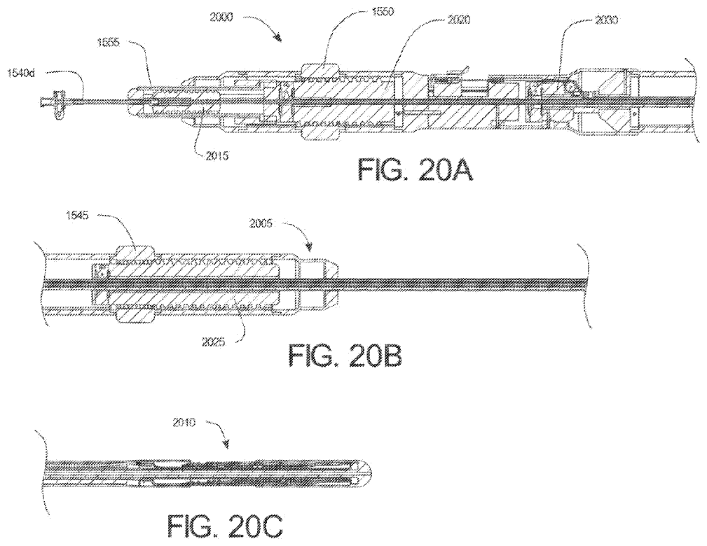

Proximal region 1505 of the handle portion 1520 may further comprise a compensation mechanism, for example an internal mechanism (described in further detail below, with reference to FIGS. 19A-C, FIGS. 18A-I, FIGS. 20A-C) that provides a leadscrew system (shown with reference to FIGS. 8A-C) that is common to both the second thumbwheel 1550 and the third thumbwheel 1555, whereby the actuation of the second thumbwheel 1550 may mechanically displace the third thumbwheel 1555. That is, actuation of the second thumbwheel 1550 may displace the second catheter 1430, the third thumbwheel 1555, and the third catheter 1445 collectively, at the same time and in the same direction because they are mechanically linked, as a system.

Expanded-view section box 1570 shows an enlarged view of the subject of detail-view section box 1565, and comprises an enlarged view of the compressed prosthetic heart valve device 1535, the distal covering section 1425 of the third catheter 1445, and the proximal covering section 1415 of the fourth catheter 1450, and is provided for clarity.

Reference is now made to FIGS. 16A-B, which are schematic illustrations of a delivery system 1500, in accordance with some applications of the invention. Further details specific to the distal region 1515, mid region 1510, and proximal region 1505 of the handle portion 1520 will be provided.

Specifically, distal handle region 1515 may further comprise a distal region handle cap 1600 which may provide a bearing surface 1605 for coupling to a holding system (not shown) and allowing relative rotation between a portion of the delivery system 1500 and the holding system. First thumbwheel 1545 can be contained within a plurality of thumbwheel covers 1610, which act to both contain the first thumbwheel 1545, and fasten cylindrical (or otherwise shaped) portions of the distal handle region 1515 together. A translation slot 1615 on the distal handle region 1515 may provide clearance for the translation of a saline flush port 1540a that controllably moves with the fourth catheter 1450, as the first thumbwheel 1545 is rotatably actuated in either a first direction or a second direction, opposite the first.

The proximal handle region 1505 may further comprise a proximal region handle cap 1630 which may provide a bearing surface 1635 for coupling to a holding system (not shown) and allowing relative rotation between a portion of the delivery system 1500 and the holding system. Second thumbwheel 1550 can be contained within a plurality of thumbwheel covers 1610, which act to both contain the second thumbwheel 1550, and fasten cylindrical (or otherwise shaped) portions of the proximal handle region 1505 together. A translation slot 1625 on the proximal handle region 1505 may provide clearance for the translation of a saline flush port 1540c that controllably moves with the second catheter 1430, as the second thumbwheel 1550 is rotatably actuated in either a first direction or a second direction, opposite the first.

With reference to FIG. 16B, mid handle region 1510 may further comprise an exit slot 1620 for a saline flush port 1540b, which can facilitate removal of entrapped air from between concentrically adjacent catheters during device preparation, as described above.

As shown, mid handle region 1510 can comprise a plurality of tether shuttles 1640 that are configured to controllably optimize tension between a prosthetic heart valve device (not shown) and a plurality of tethers (1440, FIG. 14) that are configured for connection to a portion of the prosthetic heart valve device through a clasping mechanism, detailed below. Tether shuttles 1640 may comprise a tether shuttle body 1645 and a tether shuttle latch 1650 that is configured to controllably rotate around a tether shuttle latch hinge 1655 from a first position to a second position rotationally displaced from the first, and is further configured for mechanical attachment to a proximal portion of a tether jacket 1660. By actuating the tether shuttle latch 1650, an internal connection in communication with a proximal portion of a tether jacket 1660 can withdraw the proximal portion of the tether jacket 1660 concentrically overtop of an internal tether cable (not shown) from a distal position to a proximal position opposite the distal position, thus providing controllable connection and release of the tether (1440, FIG. 14) from a portion of a prosthetic heart valve device (described further below, with reference to FIGS. 17A-F).

The tether shuttle body 1645 can be generally rectangularly shaped and can transit within a tether shuttle slot 1665 from a first end of the tether shuttle slot 1665 to a second end opposite the first. The tether shuttle body 1645 may be spring biased (not shown) in a first proximal position corresponding to the first end of the tether shuttle slot 1665, and can be translated either manually by way of pushing, or automatically such as when placed under tensile loading, transmitted along the tether (1440 FIG. 14) from the prosthetic heart valve device.

Reference is made to FIGS. 17A-F which are schematic illustrations of a prosthetic heart valve device retention region 1530 of a delivery system, in accordance with some applications of the invention. An enlarged view of a prosthetic heart valve device retention region 1530 is provided in FIG. 17A. The retention region 1530 can comprise a distal outer covering 1425 that is distally connected to a third catheter 1445 which may extend through a second catheter 1430 that may have a guidewire lumen 1760 running therethrough, and a proximal outer covering 1415 extending from a fourth catheter 1450; the distal and proximal outer coverings (1425, 1415 respectively) collectively providing location for a compressed prosthetic heart valve device 1535, as described above.

More specifically, the prosthetic heart valve device retention region 1530 can further comprise a plurality of tether connector apparatuses 1455 in a closed configuration 1700. In the closed configuration 1700, tether connector apparatus 1455 is concentric with and disposed radially adjacent to the second catheter 1430, and generally in-line with a long axis of the second catheter 1430 (axis not shown). The tether connector apparatus 1455 is schematically illustrated as being in closed and connected contact with a portion of the compressed prosthetic heart valve device 1535, and provides radial and tensile constraining force against the compressed prosthetic heart valve device 1535, thereby maintaining it in a closed and compressed configuration, suitable for delivery. More specifically, the tether connector apparatus 1455 may be in closed and connected contact with a connection element such as an atrial connection element 1720 having an atrial connection tab 1730, of the compressed prosthetic heart valve device 1535. The tether connector apparatus 1455 can be in mated contact with distal-most portions of both a tether jacket 1740, and an inner cable 1775, the relationship being schematically illustrated in FIG. 17F, with hidden lines.

More specifically, with reference to the tether connector apparatus 1455, a distal portion of the tether jacket 1740 may be in mated connection (connected through a tether connector cover sleeve 1735) with a tether connector cover 1715 that is configured to slidably mate with and internally contain a tether connector 1725; the tether connector 1725 further being in mated contact with an internal cable 1775 running within the tether jacket from a first end to a second end. A proximal portion of the tether jacket 1660, opposite the distal end may be in mated connection with an actuatable portion of a shuttling mechanism 1705, which can controllably and translationally position the tether connector cover 1715 in either a first or second position (opposite the first), relative to the internal tether connector 1725; the tether connector also being in mated connection with a fixed portion of a shuttling mechanism 1705 by way of the internal cable 1775 and configured to remain stationary.

When the tether connector cover 1715 is distally biased (first position, closed) as schematically illustrated in FIG. 17C, it can preferentially cover the tether connector 1725, thereby entrapping a portion of a compressed prosthetic heart valve device 1535, for example a connection element such as an atrial connection element 1720 having an atrial connection tab 1730. The perspective of the shuttle mechanism 1705 corresponding to this first closed position of the tether connector cover 1715 is schematically illustrated in FIG. 17B.

With reference to FIG. 17C, additional features of the second catheter 1430 are described. Specifically, a series of regions of differing stiffnesses are described. Extending from the distal end of the second catheter 1430 is a distal stiff region 1745, followed by a distal stiffness transition region 1750, and finally a distal flexible region 1755. The inherent stiffness of the distal region of the second catheter 1430 transitions from a stiffer section (1745) to the most flexible section (1755), and provides for enhanced flexibility and allowance for traversal of tight radii bends (as experienced during implantation, for example).

When the tether connector cover 1715 is proximally biased (second position, opposite the first and open) as schematically illustrated in FIG. 17E, it can preferentially reveal (indicated by arrow denoting translation 1770) the tether connector 1725, thereby releasing a portion of a compressed prosthetic heart valve device 1535, for example a connection element such as an atrial connection element 1720 having an atrial connection tab 1730. The perspective of the shuttle mechanism 1710 corresponding to this second open position of the tether connector 1725 (after rotation of the tether shuttle latches 1650, indicated by arrows 1765) is schematically illustrated in FIG. 17D.

Reference is made to FIGS. 18A-I which are a sequence of schematic illustrations depicting an expansion of a prosthetic heart valve device as deployed by a delivery system, in accordance with some applications of the invention. Turning to FIGS. 18A-B, a prosthetic heart valve device retention region 1530 is depicted, having a first closed state (FIG. 18A) with a proximal outer covering 1415 of the fourth catheter 1450 in a closed position, covering at least a portion of a compressed prosthetic heart valve device 1535. The prosthetic heart valve device retention region 1530 is also depicted having a second opened state (FIG. 18C) with the proximal outer covering 1415 of the fourth catheter 1450 in an open position and displaced proximally from the closed position a distance D1, thereby revealing a plurality of tether connector apparatuses 1700 in a closed configuration just prior to expansion of at least a portion of the compressed prosthetic heart valve device 1535 and the plurality of tether connector apparatuses 1700.

The proximal outer covering 1415 of the fourth catheter 1450 can be displaced the distance D1 through actuation of first thumbwheel 1545 as described above and in FIG. 18B (indicated by rotation arrow 1830). The proximal outer covering 1415 of the fourth catheter 1450 can also be displaced the distance D1 in an opposite direction, thereby bringing it back to the closed state (FIG. 18A) as described above, by actuating the same first thumbwheel 1545.

Once in the opened state (FIG. 18C), just prior to expansion of a portion of the compressed prosthetic heart valve device 1535 (for example, an atrial region 1410), the atrial region 1410 may have a first diameter d1. After expansion of a portion of the compressed prosthetic heart valve device 1535 (for example, the atrial region 1410) the atrial region 1410 may have a second diameter d2 larger than the first (FIG. 18D) and be in a configuration suitable for engagement with an atrial surface of a native heart (not shown). Tethers in a fully expanded state 1800 are also present in FIG. 18D.

With reference to FIG. 18E, the distal outer covering 1425 is depicted in a closed state, prior to displacement towards an open state. A partially deployed prosthetic heart valve device 1835 having a partially deployed atrial region 1805 can be further deployed by displacing the distal outer covering 1425 of the third catheter 1445 distally, by at least a distance D2 (FIG. 18G) through actuation of the third thumbwheel 1555 (represented by arrow 1865, FIG. 18F), thereby revealing at least a portion of the partially deployed prosthetic heart valve device 1835, for example, a ventricular portion in a compressed configuration 1845, and exposing coupling pegs 1820 which are configured to releasably mate with ventricular anchor coupling slots 1825. The distal outer covering 1425 of the third catheter 1445 can also be displaced the distance D2 in an opposite direction, thereby bringing it back to a closed state (FIG. 18E) as described above.

Once in the partially deployed state (FIG. 18E), but just prior to final expansion (FIG. 18G), a portion of the compressed ventricular region 1845 may have a third diameter d3. After expansion of the compressed ventricular region 1845, the deployed ventricular region 1840 may have a fourth diameter d4 larger than the third (FIG. 18G) and be in a configuration suitable for engagement with a ventricular surface of a native heart (not shown).

With reference to FIGS. 18H-I, a sequence of schematic illustrations depicting final deployment of a prosthetic heart valve device from a delivery system is depicted, in accordance with some applications of the invention.

Schematic illustrations of fully expanded atrial region 1850, fully expanded annular region 1855, and fully expanded ventricular region 1860 are presented in FIG. 181, in accordance with some applications of the invention. Fully expanded atrial region 1850 is configured for engagement with an atrial tissue surface of a native heart, for example a left atrial surface of a mitral valve (see FIGS. 5A-5B). Fully expanded annular region 1855 is configured for engagement with an annular tissue surface of a native heart, for example an annular surface of a mitral valve (see FIGS. 5A-5B). Fully expanded ventricular region 1860 is configured for engagement with a ventricular tissue surface of a native heart, for example any combination of a left ventricle, mitral valvular leaflets, and/or chordae tendineae (see FIGS. 5A-5B).

Controlled, final release and permanent implantation of the prosthetic heart valve device 1810 may be achieved by collective actuation of each of the tether shuttles 1640 (FIG. 16B, FIG. 18H) through actuation of the tether shuttle latch 1650 (FIG. 16B, FIG. 18H) of each tether shuttle 1640, thereby resulting in tether shuttles 1640 in an open configuration 1710. A fully released, permanently implanted prosthetic heart valve device 1810 is schematically illustrated in FIG. 18I, in accordance with some applications of the invention. Once each tether shuttle 1640 has been actuated and tether connectors fully opened 1815, each atrial connection tab 1730 may be released from constraint, thereby allowing each atrial region to fully expand 1850, resulting in a fully released and permanently implanted prosthetic heart valve device 1810.

With reference to FIGS. 19A-D, schematic illustrations depicting the conformational change mechanics of the second catheter and the third catheter outer covering are provided, in accordance with some applications of the invention.

Specifically, FIG. 19A describes the overall effect of the compensation mechanism within the delivery system, on the anchor structure of the prosthetic heart valve device when the partially deployed atrial region 1805 of the prosthetic heart valve device has been advanced into contact with a native atrial floor (not shown), and a seating force has been applied with the first catheter 1420, thus maintaining contact between the partially deployed atrial region 1805 and the native atrial floor (not shown). In FIG. 19A, it can be seen that the first catheter 1420 and the fourth catheter 1450 have been displaced distally, creating tension on the tethers 1920, and generating a seating force for the partially deployed atrial region 1805, due to the connections therebetween. This distal displacement of the first catheter 1420 and the fourth catheter 1450 is enabled through the compensation mechanism of the delivery system, the details of which are now described with reference to FIGS. 19B-D. As depicted in FIG. 19B, a simplified view of the distal-most portion of the delivery system, pre-displacement 1910 is provided. Embodiments of tethers and prosthetic heart valve devices are not presented in FIG. 19B, in order to more clearly schematically illustrate the mechanical interactions present during this stage of device operation, in the context of the catheters involved. Element D5 denotes a first distance between the distal-most region of the first catheter 1420, and a reference point on the second catheter, near the stiffness transition region 1750. By actuating the third thumbwheel (1550, FIG. 19C) denoted by rotation arrow 1900 (FIG. 19C), the distal retention region 1905 and partially deployed prosthetic heart valve device (not shown) are all translated proximally until a second distance, denoted by element D6, is arrived at between the distal-most region of the first catheter 1420, and the same reference point on the second catheter, near the stiffness transition region 1750. This proximally directed position (post-displacement) 1915 is described in FIG. 19D, wherein embodiments of tethers and prosthetic heart valve devices are also absent, in order to more clearly schematically illustrate the mechanical interactions present during this stage of device operation, in the context of the catheters involved. This change in position of the distal retention region 1905, activated by the compensation mechanism within the delivery system can allow for better control of the prosthetic heart valve delivery. The compensation mechanism within the delivery system can aid in controlling the conformational changes the anchor structure goes through to better approximate against the anatomical structures of the ventricle, can improve clearance between portions of the prosthetic heart valve and ventricular region structures, and is reversible in the event repositioning and re-approximation of the prosthetic heart valve is necessary.

With reference to FIGS. 20A-C, schematic illustrations depicting an embodiment of an exemplary delivery system viewed in cross-section are provided, in accordance with some applications of the invention. FIG. 20A depicts an embodiment of the mid and proximal regions of a delivery system shown in cross-section 2000. Also shown are the leadscrew 2015 of the third thumbwheel 1555, and the leadscrew 2020 of the second thumbwheel 1550. Finally, a cross-sectional depiction of the tether tension conditioning mechanism 2030 is provided.

FIG. 20B depicts an embodiment of the distal region of a delivery system shown in cross-section 2005. Also shown is the leadscrew 2025 of the first thumbwheel 1545. FIG. 20C depicts an embodiment of the retention region of a delivery system show in cross-section 2010.

While the subject of the present disclosure has been described in its preferred embodiments, it is to be understood that the words which have been used are words of description and not of limitation. Therefore, changes may be made within the appended claims without departing from the true scope of the present subject.

It will be appreciated by persons skilled in the art that the present invention is not limited to what has been particularly shown and described hereinabove. Rather, the scope of the present invention includes both combinations and sub-combinations of the various features described hereinabove, as well as variations and modifications thereof that are not in the prior art, which would occur to persons skilled in the art upon reading the foregoing description.

1-120. (canceled)

121. A prosthetic heart valve device for treating a deficient native atrioventricular valve of a heart comprising a native atrium, a native annulus and a native ventricle, the prosthetic heart valve device comprising:
an expandable valve frame forming an annular flow path extending in an axial direction, the expandable valve frame comprising:
an inflow region;
a mid-region connected to the inflow region;
an outflow region connected to the mid-region.
a plurality of leaflets mounted to the expandable valve frame to control flow through the annular flow path; and
a valve sealing cover extending across the inflow region and the outflow region and configured to prevent flow through the expandable valve frame; and
an anchoring structure configured to surround the expandable valve frame and connect to the expandable valve frame via a plurality of connection elements, the anchoring structure comprising:
an atrial region configured to be positioned within the native atrium of the heart;
an annular region (810) configured to surround the expandable valve frame, positioned within the native annulus of the heart;
a ventricular region configured to be positioned within the native ventricle of the heart;
wherein spacing between the plurality of connection elements form at least one window sized in accordance with a native valve leaflet; and
an anchor sealing cover extending over the anchoring structure to prevent flow through the anchoring structure.

122. The prosthetic heart valve device of claim 121, wherein the at least one window forms a void within the anchoring structure to avoid blood flow obstruction.

123. The prosthetic heart valve device of claim 121, wherein the at least one window comprises a space between adjacent connection elements that allows blood to flow therethrough.

124. The prosthetic heart valve device of claim 121, wherein the at least one window forms a gap in a ventricular region of the anchoring structure that is configured to not push against the native valve leaflet when the prosthetic heart valve device is expanded.

125. The prosthetic heart valve device of claim 121, wherein:
the plurality of connection elements forms a plurality of windows between adjacent connection elements.

126. The prosthetic heart valve device of claim 125, wherein each of the plurality of windows is circumferentially spaced about a perimeter of the anchoring structure at even intervals.

127. The prosthetic heart valve device of claim 125, wherein each of the plurality of leaflets is located in a window.

128. The prosthetic heart valve device of claim 125, wherein each of the plurality of connection elements comprises an axially extending member extending from the anchoring structure to connect to the expandable valve frame.

129. The prosthetic heart valve device of claim 128, wherein each of the plurality of windows is located alongside a heel structure joining the anchoring structure with the plurality of connection elements.

130. The prosthetic heart valve device of claim 129, wherein each of the plurality of windows is between adjacent heel structures.

131. The prosthetic heart valve device of claim 129, wherein each of the heel structures comprises a curved structure configured to conform with a ceiling of a ventricle of the deficient native atrioventricular valve.

132. The prosthetic heart valve device of claim 125, wherein there are three windows and three leaflets.

133. The prosthetic heart valve device of claim 125, wherein the anchoring structure further comprises a plurality of atrial conformance structures aligned with the plurality of windows.

134. The prosthetic heart valve device of claim 133, wherein each of the plurality of atrial conformance structures comprises an atrial release member for attaching to a delivery system.

135. The prosthetic heart valve device of claim 125, wherein each of the plurality of connection elements comprises a ventricular release member for attaching to a delivery system.

136. The prosthetic heart valve device of claim 125, wherein adjacent connection elements of the plurality of connection elements are connected by a leaflet attachment rail.

137. The prosthetic heart valve device of claim 136, wherein each of the leaflet attachment rails comprises a flexure geometry to allow flexure of the leaflet attachment rail.

138. The prosthetic heart valve device of claim 121, wherein the prosthetic heart valve device is configured to controllably transition between a radially compressed state configured for delivery, and a radially expanded state configured for implantation.

139. The prosthetic heart valve device of claim 121, wherein the expandable valve frame is configured to transition between a blood-flow permitting state and a blood flow preventing state.

140. The prosthetic heart valve device of claim 121, wherein:
the inflow region has a first stiffness;
the mid-region has a second stiffness; and
the outflow region has a third stiffness.

141. A method of treating a deficient native valve of a heart, the method comprising:
advancing a prosthetic heart valve in a collapsed state through vasculature of a patient using a delivery catheter;
inserting the prosthetic heart valve into an atrioventricular valve of the heart;
expanding the prosthetic heart valve into an expanded state within an atrioventricular annulus of the heart, the prosthetic heart valve comprising:
an anchoring structure having a window, the anchoring structure for attaching to the atrioventricular annulus of the heart; and
an expandable valve frame within the anchoring structure to replace native leaflets of the atrioventricular annulus;
aligning the window with a native anterior leaflet of the heart so that when the prosthetic heart valve is expanded, the native anterior leaflet of the atrioventricular annulus is able to move freely; and
releasing the prosthetic heart valve from the delivery catheter.

142. The method of claim 141, further comprising orienting the anchoring structure to prevent left ventricular outflow tract obstruction.

143. The method of claim 142, wherein aligning the window with the native anterior leaflet further comprises orienting the window with an outflow tract of the heart.

144. The method of claim 141, further comprising positioning the window so that when the prosthetic heart valve is expanded, the anchoring structure does not push against a native anterior leaflet of the heart.

145. The method of claim 144, wherein aligning the window with the native anterior leaflet further comprises aligning a prosthetic valve leaflet of the expandable valve frame with the native anterior leaflet of the atrioventricular annulus.

146. The method of claim 145, wherein aligning the prosthetic valve leaflet of the expandable valve frame with the native anterior leaflet of the atrioventricular annulus comprises positioning the prosthetic heart valve in one of three orientations to align multiple prosthetic valve leaflets of the expandable valve frame with multiple anatomic leaflets of the atrioventricular annulus.

147. The method of claim 141, further comprising viewing the native anterior leaflet of the atrioventricular annulus through a window in the anchoring structure.

148. The method of claim 147 wherein viewing the native anterior leaflet of the atrioventricular annulus through the window in the anchoring structure comprises viewing the native anterior leaflet between ventricular conformance structure support struts of the anchoring structure.

149. The method of claim 147, wherein viewing the native anterior leaflet of the atrioventricular annulus through the window in the anchoring structure comprises viewing the native anterior leaflet between valve commissure attachment regions of the expandable valve frame.

150. The method of claim 141, wherein engaging the anchoring structure of the prosthetic heart valve with the atrioventricular annulus of the heart comprises engaging a plurality of atrial conformance structures extending from an annular region of the anchoring structure with an atrial surface of the heart.

151. The method of claim 141, wherein engaging the anchoring structure of the prosthetic heart valve with the atrioventricular annulus comprises engaging a plurality of anchoring elements extending from an annular region of the anchoring structure to engage tissue by piercing tissue of the atrioventricular annulus with plurality of anchoring elements.
